(19) United States
(12) Patent Application Publication
(10) Pub. No.: US 2026/0160753 A1
(43) Pub. Date: Jun. 11, 2026

(54) THREE-DIMENSIONAL MULTILAYER MULTICELLULAR MODEL OF ENDOMETRIAL CANCER FOR HIGH THROUGHPUT DRUG SCREENING

(71) Applicant: Oregon State University, Corvallis, OR (US)

(72) Inventors: Kaitlin C. Fogg, Corvallis, OR (US); Ines Alexandra Cadena, Corvallis, OR (US); Mina Buchanan, Corvallis, OR (US); Alyssa Grace Almer, Corvallis, OR (US); Molly A. Jenne, Corvallis, OR (US); Conor Gage Harris, Corvallis, OR (US)

(73) Assignee: Oregon State University, Corvallis, OR (US)

(21) Appl. No.: 19/180,093

(22) Filed: Apr. 15, 2025

Related U.S. Application Data
(60) Provisional application No. 63/634,878, filed on Apr. 16, 2024.

(51) Int. Cl. G01N 33/50; C12N 5/09

(52) U.S. Cl. CPC G01N 33/5011; C12N 5/0693; G01N 33/5064; C12N 2513/00; C12N 2533/30; C12N 2533/52; C12N 2533/54; C12N 2533/90; C12N 2537/10

(57) ABSTRACT

A dual hydrogel system, comprising a first hydrogel that is a crosslinked gelatin hydrogel, a second hydrogel that is a crosslinked poly(ethylene glycol) hydrogel, endothelial cells intermediate and substantially coextensive with the first and second hydrogels, and endometrial cancer cells disposed on a first surface of the second hydrogel. A method for assessing the effectiveness of a therapeutic agent for the treatment of endometrial cancer using the hydrogel system.

### CROSS-REFERENCE TO RELATED APPLICATION

This application claims the benefit of U.S. Patent Application No. 63/634,878, filed Apr. 16, 2024, expressly incorporated herein by reference in its entirety.

### BACKGROUND

Endometrial cancer is the most common gynecological cancer in developed countries and the sixth most common type of cancer worldwide, presenting a significant healthcare challenge. In 2023, 66,200 women were diagnosed with this disease, and over 13,000 women died from endometrial cancer in the United States alone. Furthermore, incidence rates of endometrial cancer have been rising since the 1970s, whereas for most cancers these incidences have been declining. Although early-stage (localized) endometrial cancer can be effectively treated with surgery, chemotherapy, and radiation, advanced stages (regional, distant) often have poor prognoses, with a five-year survival rate under 20%, primarily due to metastases. Despite increasing incidence rates in the United States, treatment advancements have been limited, with no significant FDA-approved therapies since the introduction of progestin therapies in the 1970s, followed by approval of pembrolizumab and lenvatinib for recurrent or metastatic endometrial cancer in 2019. Although the combination of carboplatin and paclitaxel is a standard first-line treatment for advanced endometrial cancer, it is not specifically FDA-approved for endometrial cancer and patient response rates remain low. On average, patient response rates are 62% with overall survival times of less than three years. Thus, there is an urgent need for new therapeutic options for endometrial cancer patients.

In addition to identifying novel treatment options, adequate testing models are required to reduce the number of treatments that fail before or during the clinical trial phase. This represents a critical need for physiologically relevant and high-throughput screening platforms. High-throughput screening is a powerful tool for identifying potential therapeutic compounds, and its clinical translation depends upon the biological relevance of the in vitro models used. Traditional 2D in vitro models excel in screening large numbers of doses simultaneously; however, they fail to capture the complexity of the tumor microenvironment. Cells grown in monoculture in a flat monolayer do not exhibit the cell-cell or cell-matrix interactions present in vivo. This oversimplification in drug screening often results in data that does not translate to clinical observations, where the effectiveness of a compound in vitro is frequently overestimated due to the lack of realistic capturing of drug diffusion and penetration.

In contrast, 3D cell culture models provide a more physiologically relevant context by mimicking the architecture of tumors, better replicating the gradients of oxygen, nutrients, and signaling molecules that occur in vivo, which are crucial for understanding tumor growth, metastasis, and the response to therapies. Additionally, 3D co-culture models enable the study of more complex cellular behaviors, such as invasion through the extracellular matrix (ECM) and the formation of microvessels, critical processes in cancer metastasis, and common targets of anti-cancer therapies. The few existing 3D in vitro models of endometrial cancer, such as a novel hydrogel derived from decellularized endometrium and a supramolecular gelatin hydrogel, have demonstrated that incorporating native ECM components is critical for evaluating the cell-cell and cell-ECM interactions inherent in the endometrial cancer tumor microenvironment. However, these models lack endothelial cells, a crucial component for modeling angiogenesis, which is vital for accurately studying metastasis and therapeutic responses. Furthermore, integrating 3D models with high-throughput screening capabilities remains a challenge due to the limitations of imaging and analysis techniques and the difficulties in adapting liquid handling and assay protocols for more intricate 3D systems.

Despite the advances in existing 3D models and their integration with high-throughput screening methods noted above, a need exists for improved models that incorporate ECM components and that accurately reflect the tumor microenvironment. The present disclosure seeks to fulfill this need and provides further related advantages.

### SUMMARY

In one aspect, the disclosure provides a hydrogel system. In certain embodiments, the hydrogel system, comprises:
(a) a first hydrogel (bottom) having a first surface and an opposing second surface, wherein the first hydrogel is a crosslinked gelatin hydrogel comprising collagen I and fibrin;
(b) a second hydrogel (top) having a first surface and an opposing second surface, wherein the second hydrogel is a crosslinked poly(ethylene glycol) hydrogel comprising collagen IV, fibronectin, and laminin, wherein the second surface of the second hydrogel is adjacent the first surface of the first hydrogel;
(c) endothelial cells intermediate and substantially coextensive with the first surface of the first hydrogel and the second surface of the second hydrogel; and
(d) endometrial cancer cells disposed on the first surface of the second hydrogel.

In another aspect, the disclosure provides a method for accessing the effectiveness of a therapeutic agent for the treatment of endometrial cancer. In certain embodiments, the method comprises:
(a) contacting a hydrogel system as described herein having endometrial cancer cells in the second hydrogel; and
(b) determining the viability of the cancer cells after a predetermined period of time to access the effectiveness of the therapeutic agent for the treatment of endometrial cancer.

### DESCRIPTION OF THE DRAWINGS

The patent or application file contains at least one drawing/photograph executed in color. Copies of this patent or patent application publication with color drawing(s) will be provided by the Office upon request and payment of the necessary fee.

The foregoing aspects and many of the attendant advantages of this invention will become more readily appreciated as the same become better understood by reference to the following detailed description, when taken in conjunction with the accompanying drawings.

FIGS. 1A-1C illustrate a representative multilayer multicellular model for endometrial cancer as described herein. FIG. 1A is a schematic illustration of a representative endometrial cancer model (a representative hydrogel system as described herein with PEGDA as crosslinker in top hydrogel and GelMA as crosslinker in the bottom hydrogel) demonstrating the location of the endometrial cancer cells, endothelial cells, and the top and bottom hydrogels, as well as the components used to form the hydrogels. FIG. 1B illustrates workflow for image acquisition using a Cytation 5-cell imaging multimode reader. FIG. 1C is a schematic representation of endometrial cancer invasion depth and endothelial microvessel length calculations.

FIGS. 2A-2D present factor effect plots for each cell response as a function of Col I in the bottom hydrogel ([Col I]_{B}, dashed line), fibrinogen in the bottom hydrogel ([FG]_{B}, solid line), Col IV in the top hydrogel ([Col IV]_{T}, solid line), fibronectin in the top hydrogel ([FN]_{T}, solid line), and laminin ([LMN]_{T}, solid line). FIGS. 2A-2D compare microvessel length, endometrial cancer invasion, endothelial cell coverage, and endometrial cancer coverage, respectively. Data represented as the predicted cell response to each factor. Data represent factor effect plots generated from predictive equations for each response as a function of the four input variables while other input factors are held constant at their midlevel value. Second order multiple linear regression models for each response formed from N=45 conditions.

FIGS. 3A-3D illustrate the desirability function algorithm used to identify hydrogel formulations predicted to maximize and minimize all four of the cell phenotypic behaviors simultaneously (maximize all) and (minimize all). FIGS. 3A-3D compare phenotypic responses: microvessel length, endometrial epithelial cancer invasion, endothelial cell coverage, and endometrial epithelial cancer coverage, respectively, which were measured every 6 hours for 48 hours. Matrigel was used as a control. Time showed in the x-axis represented the point after endothelial cells and cancer cells were co-culture. * p<0.05 compared with the mean of each group. Two-way ANOVA with Tukey post-test. Data represent the mean±SD (n=4) error bars are present in all groups (some too small to be seen).

FIGS. 4A-4C compare results for or endometrial cancer cells (EC) co-cultured with human microvascular endothelial cells (hMVECs) for a representative hydrogel system as described herein, with Matrigel as a negative control. Endometrial cancer cells and hMVECs were examined in either co-culture or monoculture on either Matrigel or the optimized endometrial cancer model. FIG. 4A shows microvessel length at 12 hours. FIG. 4B shows cancer invasion at 48 hours. FIG. 4C shows a diagram demonstrating the experimental setup: M=Matrigel; EC=endometrial cancer; hMVEC=human microvascular endothelial cells. Data represent the mean±SD (n=4), * p<0.05.

FIGS. 5A-5C illustrate rheological characterization of representative hydrogel systems as described herein. FIGS. 5A-5C compare storage moduli (G′), loss moduli (G″), and phase shift angle defined as tan δ=G″/G′ for the optimized top hydrogel (Top: 1.12 mg/mL ColIV, 0.16 mg/mL fibronectin, 0.05 μg/mL laminin, 10% w/v PEGDA), the optimized bottom hydrogel (Bottom: 2.5 mg/mL Coll, 2.5 mg/mL fibrinogen, 7% w/v GelMA), and GFD Matrigel (9.2 mg/mL), respectively. Frequency sweeps were conducted from 0.1 to 100 rad/sec within the linear viscoelastic (LVE) region at a constant strain amplitude of 5%, and temperature of 37° C. Data represent the mean±SD (n=3).

FIGS. 6A-6D compare cell response between endometrial cancer cell lines. Endometrial cancer cells were co-culture with the endothelial cells in the hydrogel formulation. FIGS. 6A-6D compare microvessel length, endometrial cancer invasion, endothelial cell coverage, and endometrial cancer coverage, respectively, measured after 24 and 48 hours of co-cultured. Endothelial cells (HMVEC) were seeded on top of the hydrogel formulation for the bottom and incubated for four hours at 37° C. Then the top hydrogel formulation was added and incubated for an hour at the same conditions. Finally, the endometrial cancer cells (HEC-1A, Ishikawa, and KLE) were seeded on top and covered with media. Final constructs were imaged every three hours for 48 hours. Data represent the mean±SEM (n=4).

FIGS. 7A-7D illustrate in vitro growth-inhibitory effects of the chemotherapy drug on cell response of human microvascular endothelial cells (hMVECs) and human endometrial cancer cell lines (HEC-1A, Ishikawa, KLE). Cancer cells were seeded in 96 well plates alone (2D), and co-culture in the 3D multilayer model with hMVEC cells for 24 hours. Plates were treated with 0.008-25 μm of paclitaxel for 48 hours, cell response was evaluated at 48 hours post dosage. Each cell response is normalized to the average of the values observed in the absence of drug. Cell viability and phenotypic cell response were measured in HEC-1A cells, Ishikawa cells, and KLE cells. FIG. 7A compares results for a 2D model. FIG. 7B compares results for a representative 3D model as described herein. FIG. 7C compares microvessel formation. FIG. 7D compares cancer invasion. Data represent the mean±SD (n=4).

FIGS. 8A-8C compare cell response to paclitaxel dispensed as a free drug and encapsulated in PCL nanoparticles. Cell viability, microvessel length, and cancer invasion measured in co-culture hydrogel model with HEC-1A cells (FIG. 8A), Ishikawa cells (FIG. 8B), and KLE cells (FIG. 8C). Cells were seeded in the construct, cultured for 24 hours, then treated with the drug dispensed either as free drug (FD), or loaded in PCL nanoparticles (NP) for 48 hours, at which point cell response was evaluated. * p<0.05 compared with the mean of each group. One-way ANOVA with Tukey post-test. Data represent the mean±SD (n=4). FIGS. 8A-8C also show representative images of cell tracker labeled endothelial cells (green), and endometrial cancer cells (magenta) as 2D z-projected images at 48 hours cultured post dosage for HEC-1A cells, Ishikawa cells, and KLE cells, respectively. 22 z-slices with 85 μm separation were used to capture the 3D models. Images were taken every 6 hours for 48 hours post dosage. Scale bar 1000 μm.

### DETAILED DESCRIPTION

The present disclosure provides a significant advancement in endometrial cancer research by achieving two major milestones. First, the disclosure provides a hydrogel system that is a 3D co-culture model that simulates the ECM, stiffness, and cell-cell signaling present in the tumor microenvironment. Second, a high-throughput screening platform using the 3D hydrogel system is provided that is demonstrated to be a robust tool for preclinical drug evaluation. The findings demonstrate that the 3D hydrogel system is the first high-throughput screening platform of endometrial cancer to offer a greater understanding of endometrial cancer, capturing key behaviors such as cancer invasion, endothelial cell coverage, and microvessel formation, which are not observed in spheroids on Matrigel or traditional 2D cultures. The 3D hydrogel system described herein is demonstrated to be valuable for assessing drug responses, especially when comparing free and nanoparticle-encapsulated paclitaxel. The results underscore the importance of using 3D co-culture models to capture multifaceted aspects of cancer progression beyond cell viability, including cell-ECM interactions and cell crosstalk.

In one aspect, the disclosure provides a hydrogel system. In certain embodiments, the hydrogel system, comprises:
(a) a first hydrogel (bottom) having a first surface (top) and an opposing second surface (bottom), wherein the first hydrogel is a crosslinked gelatin hydrogel comprising collagen I and fibrin;
(b) a second hydrogel (top) having a first surface (top) and an opposing second surface (bottom), wherein the second hydrogel is a crosslinked poly(ethylene glycol) hydrogel comprising collagen IV, fibronectin, and laminin, wherein the second surface (bottom) of the second hydrogel is adjacent the first surface (top) of the first hydrogel;
(c) endothelial cells intermediate and substantially coextensive with the first surface (top) of the first hydrogel (bottom) and the second surface (bottom) of the second hydrogel (top); and
(d) endometrial cancer cells disposed on the first surface (top) of the second hydrogel (top).

The hydrogel system described herein includes a first (bottom) and a second (top) hydrogel, each having two major surfaces: a first (top) surface [120 and 220] and a second opposing (bottom) surface [110 and 210], where top and bottom refer to the major surfaces of the first and second hydrogels with respect to the fabrication and use of the dual hydrogel system. In this regard, the top surface of the first (bottom) hydrogel is adjacent the bottom surface of the second (top) hydrogel. It will be appreciated that although the top surface of the first (bottom) hydrogel is adjacent the bottom surface of the second (top) hydrogel, endothelial cells are disposed intermediate (i.e., between) the bottom surface of the second (top) hydrogel is adjacent the top surface of the first (bottom) hydrogel. Cancer cells are disposed (e.g., seeded) on the first (top) surface of the second hydrogel.

The endothelial cells intermediate the first surface of the first hydrogel and the second surface of the second hydrogel are substantially coextensive with these surfaces. That is, the endothelial cells cover substantially all of the first surface of the first hydrogel and are in contact with substantially all of the second surface of the second hydrogel. The first surface of the first hydrogel and the second surface of the second hydrogel are also substantially coextensive.

In the practice of the method described herein, the top and bottom hydrogels were optimized to mimic cell responses found in endometrial cancer. The top hydrogel supports cancer spreading and invasion, while the bottom hydrogel supports endothelial microvessel formation. Two important responses are used as targets in drug screening studies. Endometrial cancer cells and endothelial cells crosstalk in the 3D model, the cancer cells migrate towards the endothelial cells, infiltrating the microvessels formed in the bottom hydrogel.

As used herein, the term “crosslinked gelatin hydrogel” refers to a gelatin hydrogel prepared by photocrosslinking a photopolymerizable gelatin (e.g., gelatin methacrylate (GelMA)) in the presence of the other components of the first hydrogel (e.g., collagen I, fibrinogen, and thrombin). Collagen I and fibrin (formed from fibrinogen and thrombin) influence microvessel formation from endothelial cells and cancer invasion.

As used herein, the term “crosslinked poly(ethylene glycol) hydrogel” refers to a poly(ethylene glycol) hydrogel prepared by photocrosslinking a photopolymerizable poly(ethylene glycol) (e.g., poly(ethylene glycol) diacrylate (PEGDA)) in the presence of the other components of the first hydrogel (e.g., collagen IV, fibronectin, and laminin). Collagen IV, fibronectin, and laminin are ECM components specific for endometrial cancer cell growth. Methods for fabrication of a representative hydrogel system is described below (see Materials and Methods).

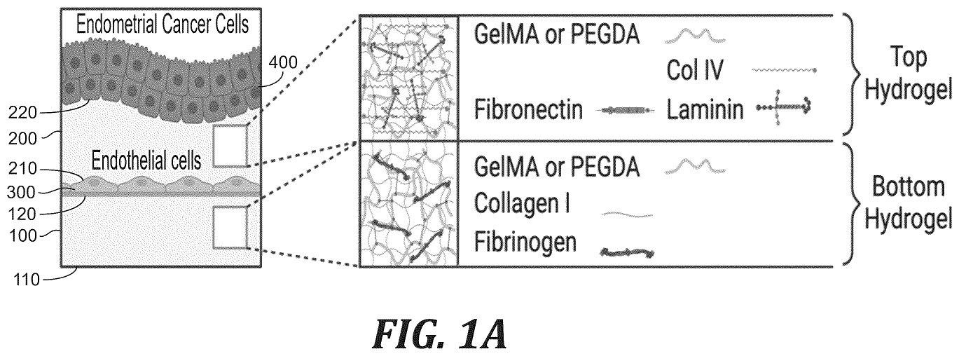

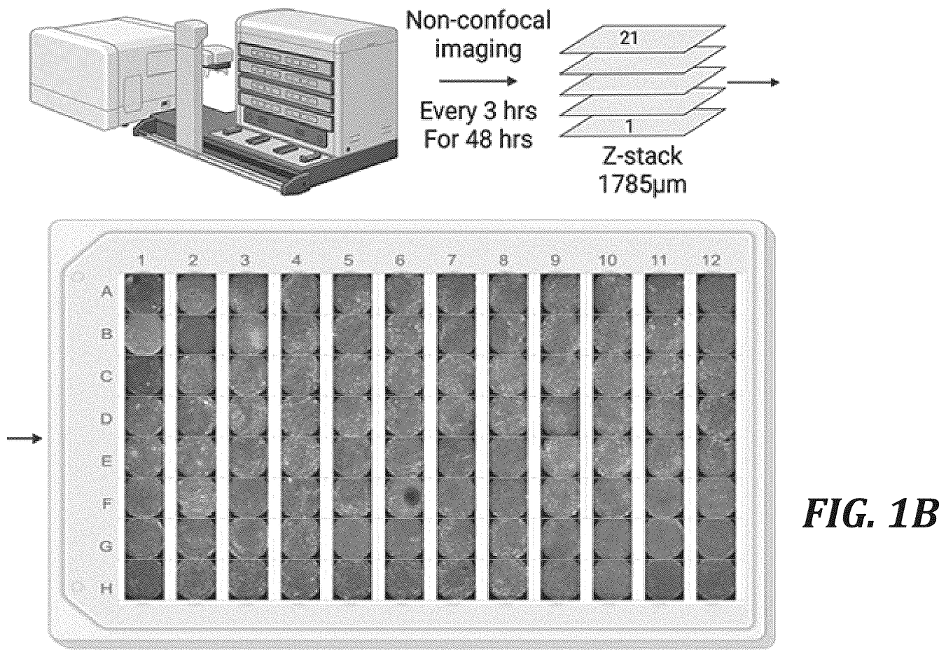

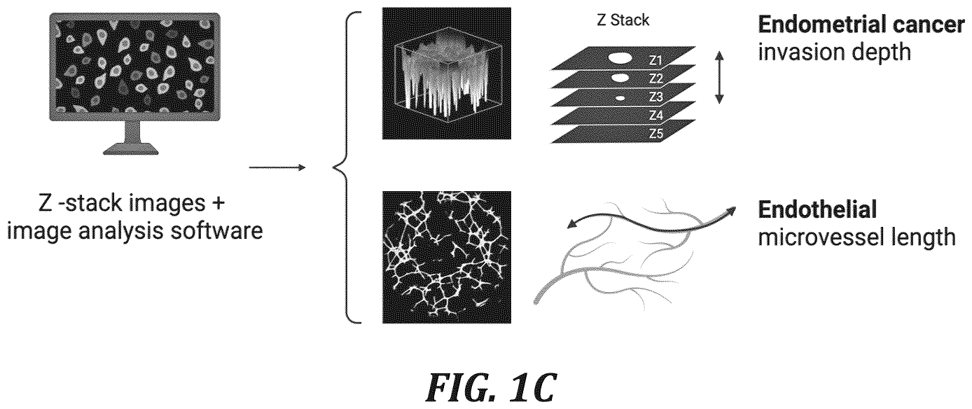

A representative hydrogel system is illustrated schematically in FIG. 1A. Referring to FIG. 1A, a representative hydrogel system comprises first hydrogel 100 having first surface 110 and second opposing surface 120, second hydrogel 200 having first surface 210 and second opposing surface 220, with endothelial cells 300 intermediate and substantially coextensive with first hydrogel second surface 120 and second hydrogel first surface 210 (second hydrogel 200 is disposed above endothelial cells 300, and cancer cells 400 disposed on second hydrogel second surface 220. The endothelial cells and cancer cells are in biological communication (cell crosstalk) through second hydrogel 200.

In the hydrogel system described herein, the endothelial cells and cancer cells are in biological communication (cell crosstalk) through the second hydrogel.

In certain embodiments, collagen I is present in the first hydrogel in an amount from about 10 to about 15 percent of the total volume of the first hydrogel. In certain embodiments, collagen I is present in the first hydrogel at a concentration of about 1.9 mg/mL, corresponding to about 12.5 percent of the total volume of the hydrogel.

In certain embodiments, fibrin is present in the first hydrogel in an amount from about 10 to about 15 percent of the total volume of the hydrogel. In certain embodiments, fibrinogen is added in the hydrogel formulation in the first hydrogel at a concentration of about 0.60 mg/mL, corresponding to about 12.5 percent of the total volume of the hydrogel.

In certain embodiments, 50% of the bottom hydrogel is GelMA.

In certain embodiments, collagen IV is present in the second hydrogel in a concentration from about 0.10 to about 0.20 mg/mL. In certain embodiments, collagen IV is present in the second hydrogel in a concentration of about 0.13 mg/mL.

In certain embodiments, fibronectin is present in the second hydrogel in a concentration from about 0.12 to about 0.18 mg/mL. In certain embodiments, fibronectin is present in the second hydrogel in a concentration of about 0.17 mg/mL.

In certain embodiments, laminin is present in the second hydrogel in a concentration from about 0.5 to about 2 μg/mL. In certain embodiments, laminin is present in the second hydrogel in a concentration of about 0.5 pg/mL.

In certain embodiments, 50% of the top hydrogel is PEGDA.

In certain embodiments, the second hydrogel has storage modulus (G′) from about 900 to about 1000 Pa. In certain embodiments, the second hydrogel has storage modulus (G′) of about 916 Pa.

In certain embodiments, the second hydrogel has storage loss modulus (G″) from about 100 to about 150 Pa. In certain embodiments, the second hydrogel has storage loss modulus (G″) of about 115 Pa.

In certain embodiments, the first hydrogel has storage modulus (G′) from about 100 to about 200 Pa. In certain embodiments, the first hydrogel has storage modulus (G′) of about 148 Pa.

In certain embodiments, the first hydrogel has storage loss modulus (G″) from about 20 to about 100 Pa. In certain embodiments, the first hydrogel has storage loss modulus (G″) of about 43 Pa.

In another aspect, the disclosure provides a method for accessing the effectiveness of a therapeutic agent for the treatment of endometrial cancer. In certain embodiments, the method comprises:
(a) contacting a hydrogel system as described herein having endometrial cancer cells in the second hydrogel; and
(b) determining the viability of the cancer cells after a predetermined period of time to access the effectiveness of the therapeutic agent for the treatment of endometrial cancer.

In certain embodiments, the method further comprises determining the cancer cell invasion depth after the predetermined period of time.

In other embodiments, the method further comprises determining the reduction of endothelial microvessel length after the predetermined period of time.

In the methods, a predetermined period of time is the amount of time sufficient for the therapeutic agent to diffuse into the second hydrogel and contact the endometrial cancer cells.

The following is a description of the design, preparation, and use of representative hydrogel systems described herein (i.e., three-dimensional multilayer multicellular models for endometrial cancer).

### Design of Experiments for Developing a 3D In Vitro Model for Endometrial Cancer

Using a design of experiments (DOE) approach, a hydrogel formulation was engineered to support human microvascular endothelial cells (hMVEC) and a hydrogel formulation was engineered to support endometrial cancer cells. Using a D-optimal experimental design, seven input variables generated 45 unique hydrogel combinations with a centrally repeated condition. Specifically, the influence of the concentration of the natural polymers Col I, fibrin, and the synthetic polymers PEGDA (10% w/v) or GelMA (7% w/v) in the bottom hydrogel layer; and the natural polymers Col IV, fibronectin, laminin, and the synthetic polymers PEGDA (10% w/v) or GelMA (7% w/v) in the top hydrogel layer were evaluated. From the experimental results, multivariant models were generated for four phenotypic responses, including microvessel length, endometrial invasion depth, endothelial cell coverage, and endometrial cancer coverage, with predicted vs. observed R² values of 0.89, 0.89, 0.70, and 0.83, respectively, and Q² values ≥0.50 suggesting that the number of model terms adequately fit the model. The R²-adjusted values remain robust and substantiate the model's reliability. Finally, the G-efficiency of the model was 51.32%. Robust model designs have a G-efficiency of 50% or larger. The ANOVA table indicated that one or more input variables significantly influenced each cell type in each hydrogel layer (p<0.05), and the interactions were described as linear or two-factor interactions (Table 1).

TABLE 1
ANOVA table for the DOE model and interplay between input and output variables with their
corresponding p-value. R² = coefficient of correlation. Q² = model predictive
power. Confidence level 95%. Non-significant values are stated with a slash (—).
Four replicates per condition were included in the experiment (n = 4 for N = 45 conditions).

| | | | | |
|---|---|---|---|---|
| | Endothelial | Endometrial | Endothelial | Endometrial |
| | microvessel | cancer | cell | cancer |
| Response | length | invasion | coverage | coverage |

p-values

| | | | | |
|---|---|---|---|---|
| model | <0.0001 | <0.00001 | <0.0001 | <0.0001 |
| Col1_{B} | — | — | 0.0015 | 0.024 |
| FG_{B} | — | — | — | 0.000022 |
| Col IV_{T} | 0.0107 | 0.0017 | — | — |
| FN_{T} | 0.00001 | — | — | 0.0072 |
| LMN_{T} | — | 0.0214 | — | 0.000012 |
| Syn_{T} (PEGDA) | — | 0.0346 | 0.0044 | — |
| Syn_{T} (GelMA) | — | 0.0346 | 0.0044 | — |
| Syn_{B} (PEGDA) | — | — | 0.0001 | 0.0120 |
| Syn_{B} (GelMA) | — | — | 0.0001 | 0.0120 |
| Col1_{B}² | — | 0.0153 | — | 0.0046 |
| FG_{B}² | 0.0080 | 0.0054 | — | — |
| ColIV_{T}² | — | 0.0164 | — | — |
| FN_{T}² | — | 0.0012 | 0.0128 | — |
| LMN_{T}² | 0.0067 | — | 0.0054 | 0.0098 |
| ColIV_{T} * Col1_{B} | — | — | 0.0150 | — |
| ColIV_{T} * FG_{B} | — | — | 0.0245 | — |
| ColIV_{T} * LMN_{T} | — | — | — | 0.0133 |
| FN_{T} * FG_{B} | — | 0.0001 | — | — |
| FN_{T} * Col1_{B} | 0.00002 | 0.0110 | — | — |
| FN_{T} * LMN_{T} | 0.0025 | — | — | — |
| FG_{B} *LMN_{T} | 0.0271 | 0.0019 | — | — |
| Col1_{B} * LMN_{T} | — | 0.0180 | — | — |
| FG_{B} * Syn_{T} (PEGDA) | 0.00001 | — | 0.0336 | — |
| FG_{B} * Syn_{T} (GelMA) | 0.00001 | — | 0.0336 | — |
| FG_{B} * SynB (PEGDA) | 0.0001 | 0.0290 | — | — |
| FG_{B} * SynB (GelMA) | 0.0001 | 0.0290 | — | — |
| Col1_{B} * Syn_{B} (PEGDA) | 0.0203 | — | — | — |
| Col1_{B} * Syn_{B} (GelMA) | 0.0203 | — | — | — |
| Col1_{B} * Syn_{T} (PEGDA) | — | — | — | 0.0306 |
| Col1_{B} * Syn_{T} (GelMA) | — | — | — | 0.0306 |
| ColIV_{T} * Syn_{T} (PEGDA) | — | 0.0362 | — | — |
| ColIV_{T} * Syn_{T} (GelMA) | — | 0.0362 | — | — |
| ColIV_{T} * Syn_{B} (PEGDA) | 0.0452 | — | — | — |
| ColIV_{T} * Syn_{B} (GelMA) | 0.0452 | — | — | — |
| FN_{T} * Syn_{B} (PEGDA) | — | — | — | 0.0090 |
| FN_{T} * Syn_{B} (GelMA) | — | — | — | 0.0090 |
| FN_{T} * Syn_{T} (PEGDA) | 0.0002 | 0.0006 | — | 0.0018 |
| FN_{T} * Syn_{T} (GelMA) | 0.0002 | 0.0006 | — | 0.0018 |
| LMN_{T} * Syn_{T} (PEGDA) | — | 0.0023 | — | — |
| LMN_{T} * Syn_{T} (GeIMA) | — | 0.0023 | — | — |
| LMN_{T}* Syn_{B} (PEGDA) | 0.0000002 | — | — | — |
| LMN_{T} * Syn_{B} (GelMA) | 0.0000002 | — | — | — |
| Syn_{T} (PEGDA) * Syn_{B} (PEGDA) | — | 0.0061 | — | — |
| Syn_{T} (PEGDA) * Syn_{B} (GelMA) | — | 0.0061 | — | — |
| Syn_{T} (GelMA) * Syn_{B} (PEGDA) | — | 0.0061 | — | — |
| Syn_{T} (GelMA) * Syn_{B} (GelMA) | — | 0.0061 | — | — |
| Lack of fit | 0.111 | 0.682 | 0.017 | 0.004 |

Validation metrics

| | | | | |
|---|---|---|---|---|
| Degree of Freedom (DF) | 24 | 17 | 29 | 28 |
| R² | 0.89 | 0.89 | 0.70 | 0.83 |
| R²-adjusted | 0.81 | 0.72 | 0.6 | 0.74 |
| Q² | 0.58 | 0.39 | 0.27 | 0.56 |

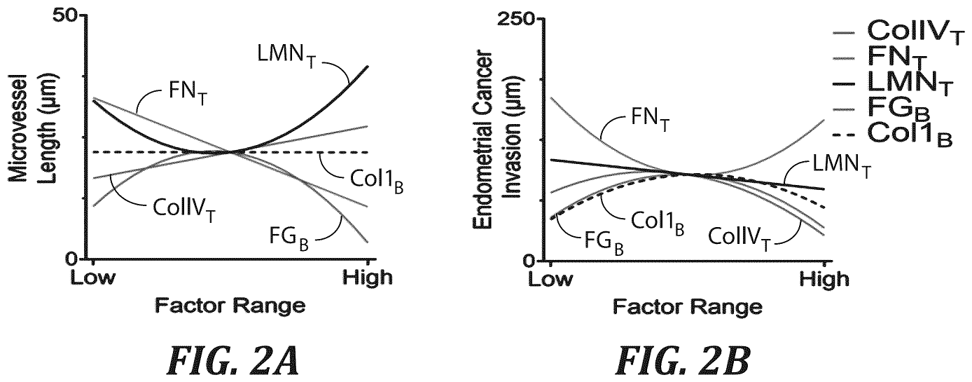

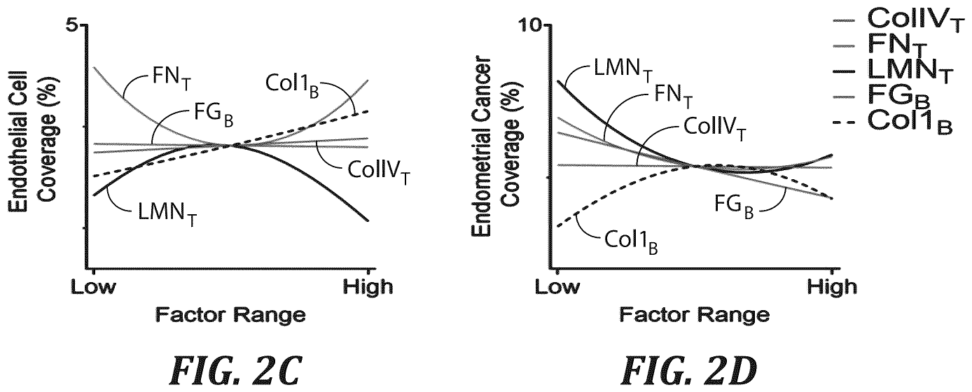

These results underscore the significant correlations between the input variables and the measured responses. Endothelial microvessel length ranged from 16 μm to 40 μm and was significantly influenced by the individual concentration of extracellular matrix components and their quadratic terms (FIG. 2A).

In the bottom hydrogel, Col IV had a positive linear effect, and the quadratic term did not contribute to microvessel formation. Laminin had a more significant influence on endothelial cell microvessel length when combined with a synthetic polymer and positively influenced microvessel length in a quadratic relationship. The concentration of fibrin in the bottom hydrogel did not have a significant effect on microvessel length alone. However, it did have a quadratic interaction, which negatively influenced this cell response (FIG. 2A). Col I in the bottom layer only showed a significant interaction with PEGDA and GelMA. All the ECM components had significant interactions with the synthetic polymers in the top and bottom layers. However, PEGDA or GelMA alone and their quadratic interaction did not influence microvessel length.

Endometrial cancer invasion was evaluated with the HEC-1A cell line as the model endometrial carcinoma cell line, ranged from 34 μm to 169 μm and was mostly influenced by the quadratic interactions in the top and bottom hydrogel layers (FIG. 2B). Col IV, fibrin, and Col I had negative quadratic relationships with endometrial cancer cell invasion, with the maximum cancer cell invasion occurring at the median concentration of these ECM components fibronectin had a positive quadratic influence. Moreover, it showed only significant interactions with the ECM components in the bottom hydrogel layer and the synthetic polymers in the top hydrogel layer but not with the synthetic polymers in the bottom layer or Col IV or Laminin (Table 2). Laminin was the only polymer that showed a negative linear relationship with endometrial cancer cell invasion. Lastly, the synthetic polymers in the top and bottom hydrogels all had significant interactions with each other (Table 2).

Endothelial cell coverage was evaluated with the hMVEC cell line and ranged from 3% to 4.2%. Col I in the bottom hydrogel had a positive linear relationship with endothelial cell coverage (FIG. 2C). Col IV and fibrin in the top hydrogel had a significant positive influence on endothelial cell coverage. Yet, their individual terms were not significant to this cell response. The quadratic influence of laminin in the top layer negatively affected endothelial cell coverage; meanwhile, the quadratic influence of fibronectin in the top layer positively contributed to endothelial cell coverage. Replacing the synthetic polymer in the bottom layer from PEGDA to GelMA increased endothelial cell coverage at low concentrations of fibrin in the bottom layer and a mid-range concentration of Col IV in the top layer.

Endometrial cancer coverage ranged from 3% to 8% (FIG. 2D). All the ECM individual components had a significant effect on cancer coverage except for Col IV in the top hydrogel layer. Interestingly, laminin and fibronectin in the top layer had a negative quadratic influence on cell coverage. Fibronectin interacted with the synthetic polymers in the top and bottom hydrogel layers, with a stronger influence in the top hydrogel layer. By increasing fibronectin in the top layer from 0.125 mg/mL to 0.175 mg/mL, the surface response curves showed a maximum endometrial cancer cell coverage at high concentrations of Col IV and fibrin and a minimum response at low Col IV and low fibrin. The quadratic influence of laminin in cancer coverage showed that by increasing this factor, the coverage decreased from 12.8% to 8.3%. Overall, these results showed that endothelial cell response and endometrial cancer response were influenced by the linear and quadratic interactions from the D-Optimal design and opposite cell responses were exhibited when one or two components were changed in the model. Therefore, achieving the ideal hydrogel formulations requires finding a delicate equilibrium between the responses of both cell types.

### Validation of DOE Model

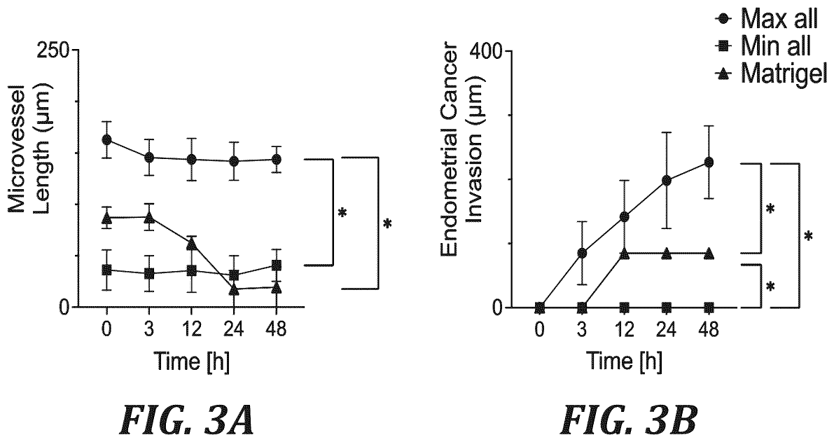

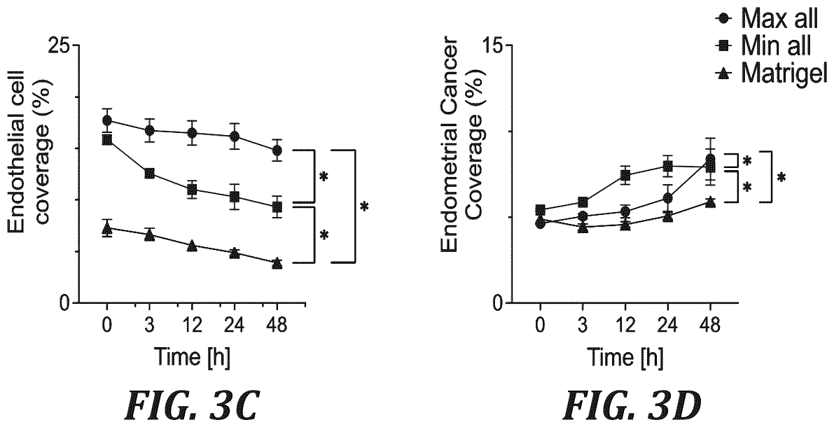

The prediction algorithm from MODDE Pro was used to find the hydrogel formulations that would maximize or minimize endothelial cell microvessel length, endometrial cancer invasion, endothelial cell coverage, and endometrial cancer coverage simultaneously (Table 1). The design criteria were based on the formulation with a lower probability of failure (less than 10%), and Matrigel was included as a control. Using the prediction tool, three points from the design space were evaluated that met the criteria to maximize microvessel length, cancer invasion and cell coverage. From these results, the hydrogel formulation was selected that favored all the cell responses simultaneously and compared it with the hydrogel formulations that minimized all cell responses as well as a Matrigel control (FIGS. 3A-3D). Additionally, the hydrogel formulations that maximized or minimized microvessel length or endometrial cancer invasion individually were evaluated.

The hydrogel formulation that maximized all cell responses (Max All) had a fibrin concentration of 0.6 mg/mL, Col I of 1.91 mg/mL, and these were mixed with GelMA (7% w/v) to form the bottom hydrogel layer. The top layer had a concentration of 0.13 mg/mL of Col IV, 0.17 mg/mL of fibronectin, and 0.50 μg/mL of laminin, and were mixed with PEGDA (10% w/v). Compared with other hydrogel formulations, the microvessel length was significantly higher, and both endometrial cancer cell invasion and endometrial cell coverage increased (FIGS. 3A-3D). Cells cultured in Matrigel exhibited a decrease in microvessel length after 12 hours, whereas the Max All formulation demonstrated stability in microvessel length, maintaining consistent values over 48 hours (FIG. 3A). This hydrogel also exhibited a higher endometrial cancer invasion and increasing values over time, compared to other hydrogel formulations, including Matrigel (FIG. 3B). The Max All hydrogel formulation showed significant differences between Matrigel and the formulation predicted to minimize all cell responses (Min All formulation).

The Min All formulation was set as a target for the prediction of the concentration ranges and synthetic polymer that will have the lowest microvessel length, endometrial cancer invasion, and cell coverage. The microvessel length of hMVEC cells was significantly lower in the Min All and Matrigel cultures compared with the Max All formulation (FIG. 3A). The Min All formulation had significantly less endometrial cancer cell invasion compared with either Matrigel or the Max All formulation (FIG. 3B). Furthermore, endothelial cell coverage was significantly lower in Min All compared with Max All and it was still significantly higher than Matrigel (FIG. 3C). Endometrial cancer coverage increased over the 48 hours across all hydrogel formulations, and the Max All formulation displayed a significantly higher cancer coverage compared to the other hydrogel formulations (FIG. 3D). Furthermore, endothelial cell coverage decreased continuously with this hydrogel, maintaining levels lower than those observed with the Max All formulation after 48 hours of culturing (FIG. 3C).

Regarding endometrial cancer coverage, the Min All hydrogel initially experienced an increase within the first 12 hours. However, following this initial increase, the response remained constant for the subsequent 48 hours, unlike other hydrogel formulations that continually increased cancer coverage throughout the 48-hour experiment (FIG. 3D). Due to the limited dynamic range of cell coverage, optimizing microvessel length and cancer invasion was the focus of further experiments. The DOE-predicted model using hydrogel formulations designed to both maximize and minimize microvessel length and endometrial cancer invasion separately was further validated. Having validated that the Max All formulation achieved the engineering objective of maximizing cell coverage, microvessel length, and endometrial cancer invasion simultaneously, this formulation is referred to herein as the representative endometrial cancer model.

### Replicating the Tumor Microenvironment of Endometrial Cancer

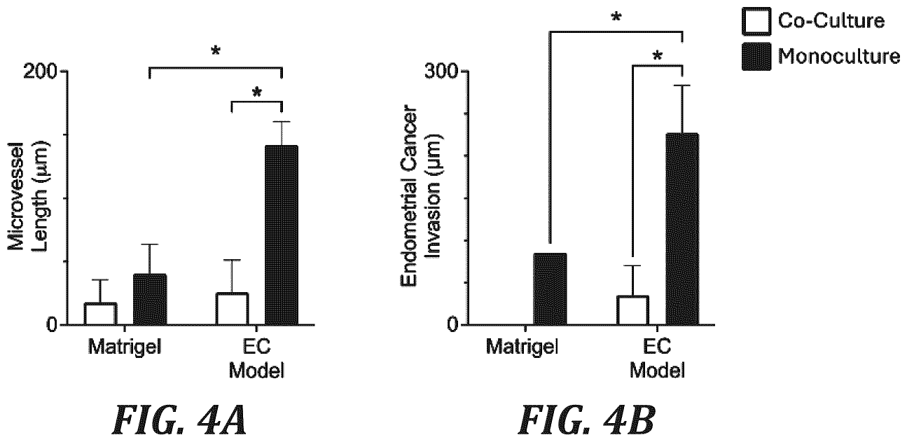

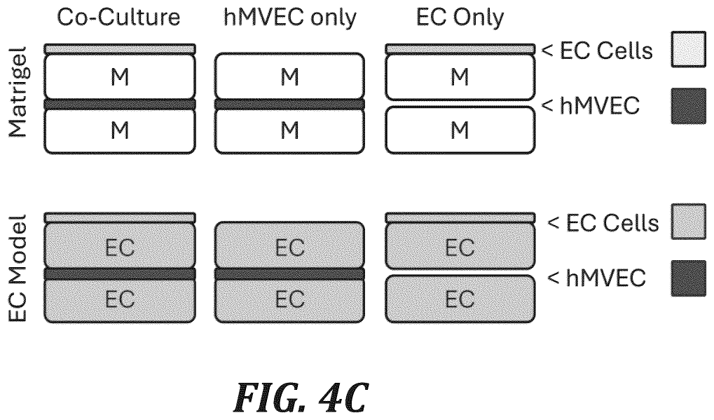

The impact of crosstalk between endothelial cells and endometrial cancer cells on microvessel formation and cancer invasion was evaluated (FIGS. 4A-4C). Each cell type was cultured on the optimized endometrial cancer model within a coculture system containing both hMVEC endothelial cells and HEC-1A, Ishikawa, or KLE endometrial adenocarcinoma cells. All four cell lines were also cultured in a monoculture system, with only one cell type present. As a control, Matrigel replaced the top and bottom hydrogel layers. On both Matrigel and the optimized endometrial cancer model the phenotypic responses were elevated for the coculture compared to the monoculture. Specifically, the microvessel length was significantly affected by the hydrogel formulation and cell crosstalk, with coculture resulting in four times higher microvessel length on the optimized endometrial cancer model compared to Matrigel. Similarly, endometrial cancer invasion was significantly influenced by the hydrogel formulation and cell crosstalk, with three times higher endometrial cancer invasion in the coculture model with the optimized endometrial cancer model.

The monoculture models had significantly greater microvessel length and invasion compared with Matrigel, indicating that the optimized hydrogel formulations overall perform better than Matrigel (FIGS. 4A and 4B).

### Comparative Rheological Analysis of the Hydrogels

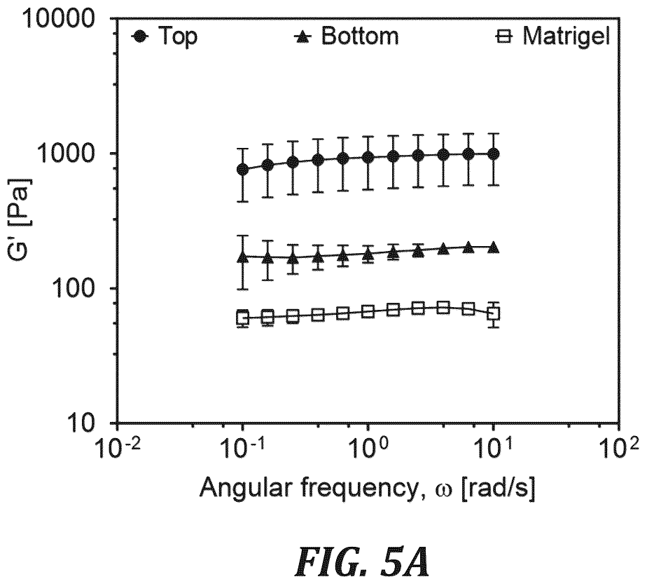

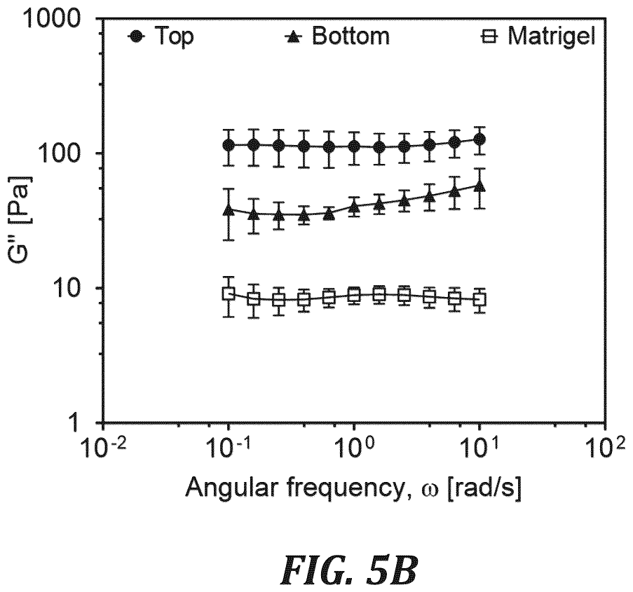

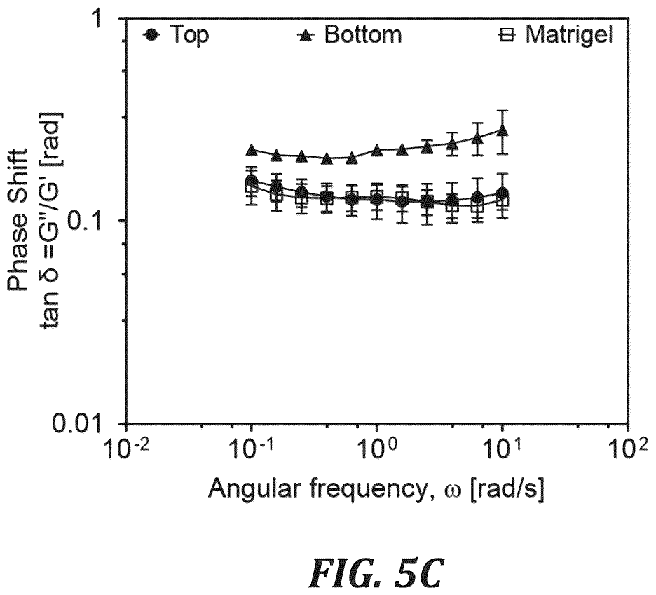

The viscoelastic properties of both layers of the representative endometrial cancer model was compared with Matrigel to identify how variations in the viscoelastic properties influence cell phenotypic responses, specifically endothelial cell microvessel formation and endometrial cancer cell invasion. Using a rheometer, the elastic modulus (G′) and loss modulus (G″) were measured to discern differences in G′ and G″ among Matrigel (9.2 mg/mL), the top hydrogel formulation (0.13 mg/mL Col IV, 0.17 mg/mL fibronectin, 0.50 μg/mL laminin, 10% w/v PEGDA), and the bottom hydrogel formulation (1.91 mg/mL Col I, 0.60 mg/mL fibrin, 7% w/v GelMA) (FIGS. 5A-5C).

The elastic modulus (G′) is the material's capacity to store energy during deformation and return to its original shape after stress removal. The loss modulus (G″) is the material's capacity to dissipate energy as heat under deformation, indicating its viscous characteristics. In the analysis, the top hydrogel demonstrated significantly higher G′ values than the bottom hydrogel and Matrigel, with Matrigel exhibiting the lowest G′ (FIG. 5A). The top hydrogel also had the highest G″ value. However, there were no significant differences between the top and bottom hydrogels or Matrigel (FIG. 5B). Throughout the frequency sweep analysis, the G′ and G″ values of the top hydrogel and Matrigel remained relatively constant. Conversely, the bottom hydrogel layer demonstrated a slight increase in G″ towards the end of the analysis. To further understand the viscoelastic behavior of the hydrogels, the phase shift angle, represented by tan δ=G″/G′, was evaluated. The top hydrogel exhibited phase shift angles comparable to Matrigel. Conversely, the bottom hydrogel displayed a higher phase shift angle, indicating a more pronounced viscous behavior within this hydrogel. While distinctions were observed between the top, bottom, and Matrigel, all tan 6 values were below 1, signifying the retention of viscoelastic properties under shear stress (FIG. 5C).

### Phenotypic Cell Response Among Different Endometrial Cancer Cell Lines

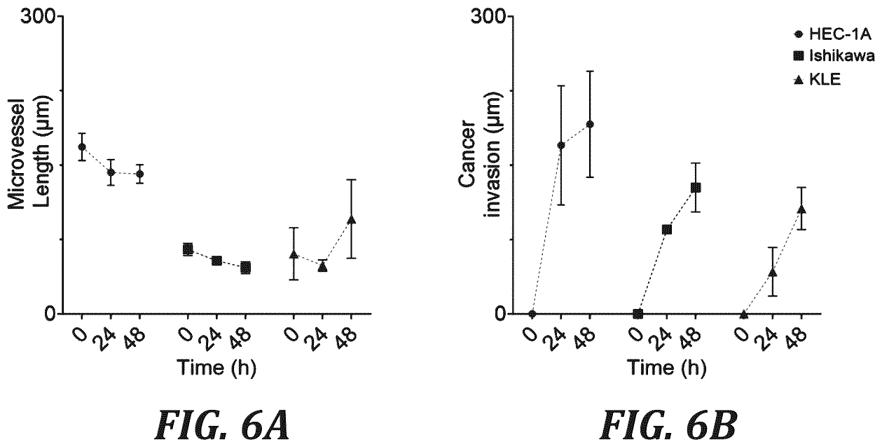

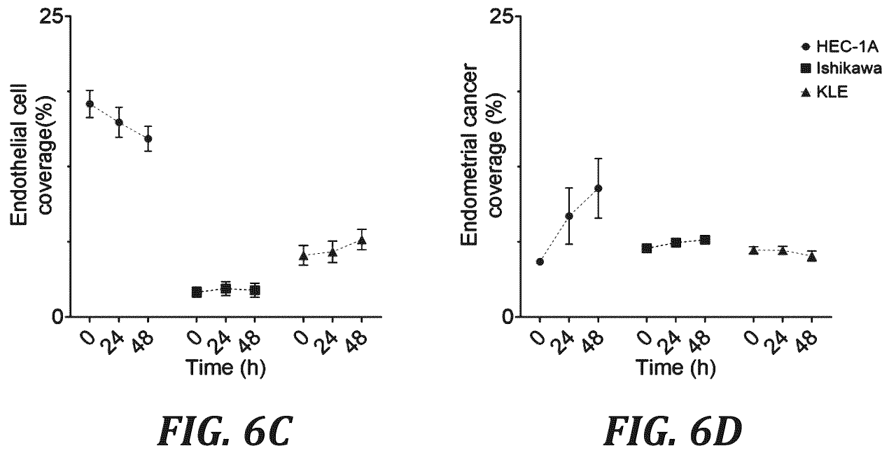

To further assess the potential of the representative multilayer hydrogel model, a comparative analysis of phenotypic cell responses using all three endometrial cancer cell lines seeded on either a representative hydrogel system as described herein (endometrial cancer model) or Matrigel was conducted. The original model was developed and validating using only HEC-1A cells, and this allowed for testing the model against other endometrial cancer cells. Specifically, hMVEC cells were seeded on the optimized bottom hydrogel layer or and co-cultured with either HEC-1A, KLE, or Ishikawa endometrial cancer cell lines seeded on the top hydrogel layer (FIGS. 6A-6D).

In co-culture with Hec-1A and Ishikawa cells, microvessel length decreased over the 48-hour culture period; however, in co-culture KLE cells exhibited a decrease in microvessel length at 24 hours, followed by an increase after 48 hours (FIG. 6A). Endometrial cancer invasion increased at all time points for all the cell lines (FIG. 6B). HEC-1A cells demonstrated the highest level of invasion, followed by Ishikawa cells and KLE cells. There were only significant differences at 24 hours between the constructs with HEC-1A cells and KLE cells. However, after 48 hours of culture, there were no significant differences in invasion depth across all the cell lines.

Endothelial cell coverage differed based upon the endometrial cancer cell line present in co-culture (FIG. 6C). Constructs with HEC-1A cells had significantly greater endothelial cell coverage compared to all other endometrial cancer cell lines across all time points, highlighting their unique impact on the endothelial cell environment. Interestingly, endothelial cell coverage decreased over time with the HEC-1A cells in co-culture; however, endothelial cell coverage increased over time for Ishikawa and KLE cells in co-culture (FIG. 6C). Notably, endometrial cancer cell coverage of HEC-1A cells increased over time; however, cell coverage of Ishikawa and KLE cells remained relatively constant (FIG. 6D). Ishikawa cells exhibit a slight increase in coverage at 24 hours, followed by a decrease at 48 hours. In contrast, KLE cells displayed a consistent increase in endothelial cell coverage over time. There were only significant differences at the 48-hour mark between the models with HEC-1A cells and the other cell lines. However, models containing KLE and Ishikawa cells showed no significant differences at 48 hours.

### Comparative Dose-Response Analysis of Paclitaxel

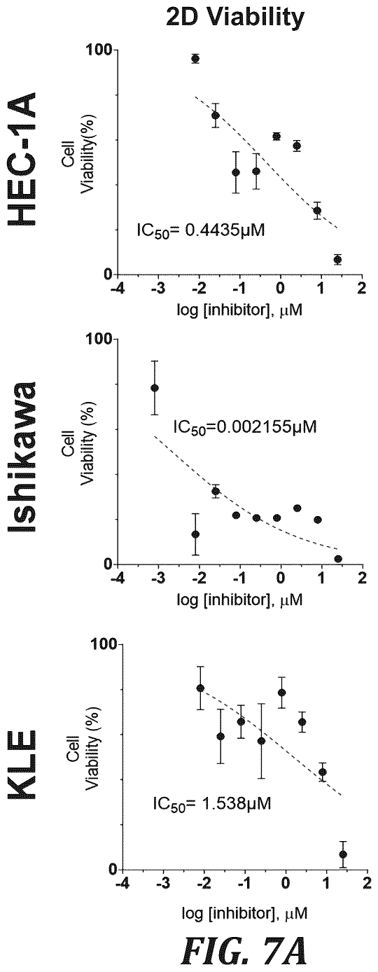

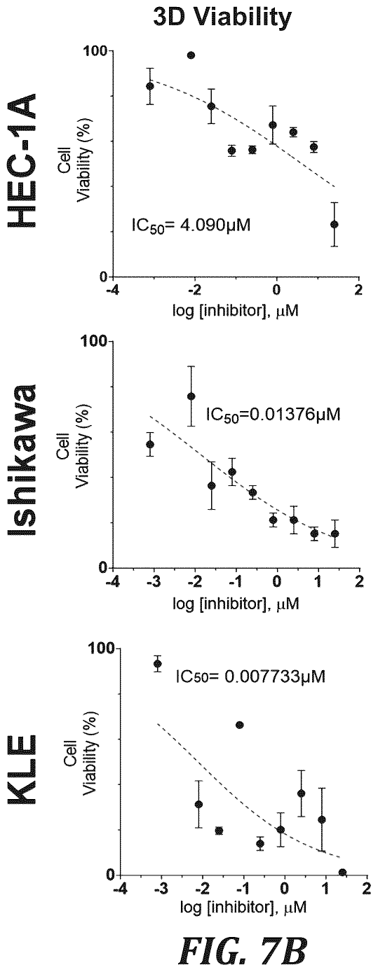

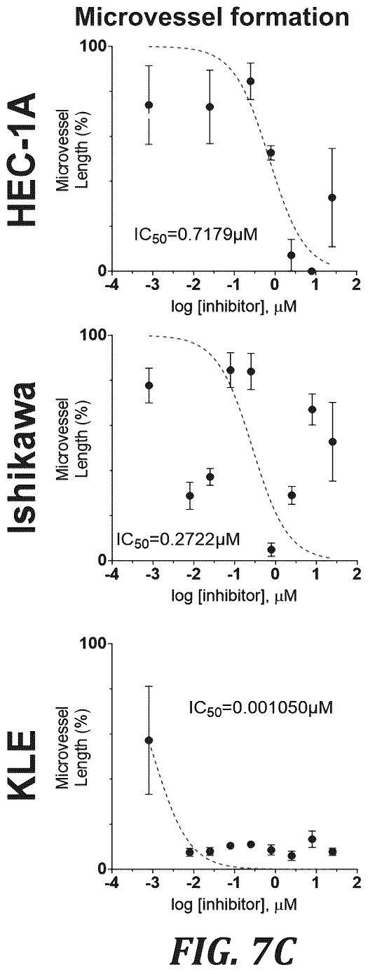

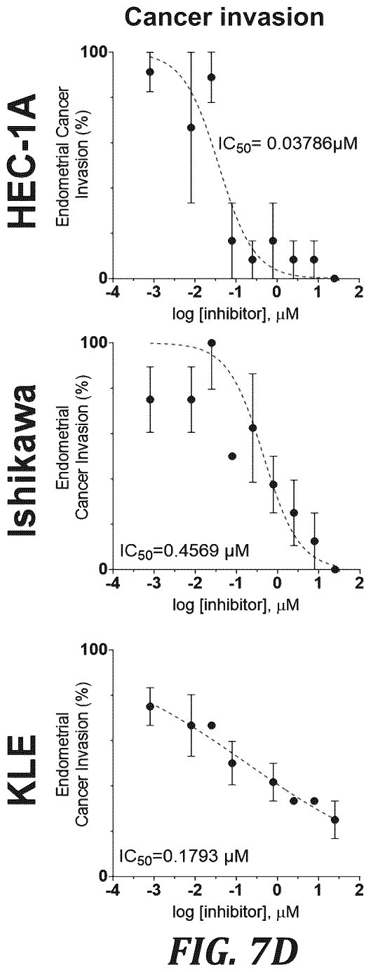

A comparative analysis was conducted of the response of three endometrial cancer cell lines to paclitaxel (a widely used chemotherapy drug, including as a first-line treatment for endometrial cancer). The inhibitory concentration of paclitaxel on the cell viability in traditional tissue culture plastic (2D), and the inhibitor concentration of paclitaxel on the cell viability and phenotypic responses within the representative multilayer hydrogel model (3D) were compared. The focus was on assessing the impact of paclitaxel on cell viability and phenotypic responses after drug exposure at 24 hours and 48 hours (FIGS. 7A-7D) through an eight-point half-log dose-response assessment. Overall, for all cell lines, the dose response-inhibition curves demonstrated increased fit to the IC50 model at 48 hours versus 24 hours.

Cell viability in the 2D models showed differences in the IC₅₀ values across the cell lines after 48 hours of culturing (FIG. 7A). The highest IC₅₀ value was observed in the KLE cells, and the lowest IC₅₀ value was observed in Ishikawa cells. Cell viability in the 3D models exhibited a different trend, revealing higher resistance to paclitaxel in HEC-1A and Ishikawa cells compared to KLE (FIG. 7B). At 48 hours, the HEC-1A cells exhibited a higher IC₅₀ value for microvessel length to Ishikawa and KLE cells (FIG. 7C). Conversely, Ishikawa cell models demonstrated a less defined fit in the dose-response curve for microvessel formation, displaying similar IC₅₀ values at both 24 and 48 hours. In contrast, the KLE cell models depicted a consistent dose-response curve in microvessel formation, aligning closely with cell viability in 3D at the 48-hour mark (FIG. 7C).

Endometrial cancer invasion demonstrated the best fit dose-response curves across all evaluated metrics (FIG. 7D). Among the cell lines, the paclitaxel-insensitive HEC-1A cells demonstrated the lowest IC₅₀ value in cancer invasion across cell lines. In contrast, the paclitaxel-insensitive Ishikawa cells displayed the highest IC₅₀ value compared to the other cancer cell lines and to both 2D and 3D cell viability results. Conversely, the paclitaxel-sensitive KLE cells exhibited a more favorable fit in the dose-response curve for endometrial cancer invasion in comparison to cell viability and microvessel formation metrics (FIG. 7D). After 24 hours of drug exposure, endometrial cancer cells HEC-1A and KLE still showed paclitaxel resistance, reflected in the poor fit of the dose-response curve for cancer invasion, a response that notably improved after 48 hours of exposure. Endothelial cell coverage and endometrial cancer coverage were also evaluated with paclitaxel after 24 hours and 48 hours of dosage. However, these metrics also exhibited poor fit to the dose-response curve. The IC₅₀ values of paclitaxel-loaded PCL nanoparticles on the three endometrial cancer cell lines were evaluated. Similar to the free drug, the dose-response curves at 48 hours displayed improved fit compared to the 24-hour curves. At both time points, KLE exhibited the highest IC₅₀ value, similar to observed 2D viability with free drug. At 48 hours, HEC-1A cells exhibited increased chemo-resistance when treated with the free drug compared with the PCL nanoparticles. However, Ishikawa cells demonstrated a higher IC₅₀ indicative of less chemo-resistance when exposed to paclitaxel encapsulated in PCL nanoparticles. Moreover, the paclitaxel-sensitive KLE cells displayed a higher IC₅₀ value at 48 hours with both dosing methods compared with the free drug.

Comparison of Cell Response to Paclitaxel Delivered as a Free-Drug Vs. Encapsulated in PCL Nanoparticles in 3D In Vitro Model

Using a representative hydrogel system as described herein (endometrial cancer model), a comparative study was conducted to analyze cell viability and phenotypic cell responses with paclitaxel, administered as both a free drug and encapsulated in PCL nanoparticles. Endothelial and endometrial cancer cells were seeded into the 3D model and cultured for 24 hours before treatment. Based on the earlier experiments, the highest IC₅₀ value for each cell line at all time points was chosen (Table 2), determining the paclitaxel concentration applied atop the constructs.

TABLE 2
Summary of IC₅₀ values for the endometrial cancer cell lines after 24 hours and
48 hours of exposure to paclitaxel. The drug was delivered as free and encapsulated
in PCL nanoparticles on top of our 2D and 3D in vitro models. Abbreviations:
cancer invasion (INV), microvessel length (MV), endothelial cell coverage (ECCC),
cancer cell coverage (CCC), polycaprolactone nanoparticles (PCL np)

| | | | | | | IC₅₀ | IC₅₀ | IC₅₀ |
|---|---|---|---|---|---|---|---|---|
| Time | | IC₅₀ | IC₅₀ | IC₅₀ | IC₅₀ | viability | viability | 2D |
| (h) | Cell line | INV | MV | ECC | CCC | 3D | 2D | PCL np |
| 24 | HEC-1A | 9.1E−03 | 4.1E−01 | 2.7E−03 | 5.6E−01 | 4.0E+00 | 7.1E−02 | 2.7E−03 |
| | Ishikawa | 2.9E−02 | 1.3E−01 | 2.0E+00 | 6.8E−01 | 7.8E−02 | 4.3E+00 | 1.9E−03 |
| | KLE | 1.2E−03 | 6.5E−01 | 6.0E−03 | 4.1E+00 | 5.7E+00 | 8.8E+00 | 8.4E−03 |
| 48 | HEC-1A | 3.8E−02 | 7.2E−01 | 2.3E−03 | 2.1E+00 | 4.1E+00 | 4.4E−01 | 3.6E−02 |
| | Ishikawa | 4.6E−01 | 2.7E−01 | 2.9E+00 | 8.8E−01 | 1.4E−02 | 2.2E−03 | 1.6E−01 |
| | KLE | 1.8E−01 | 1.1E−03 | 1.2E+00 | 2.8E−01 | 7.7E−03 | 1.5E+00 | 1.0E+00 |

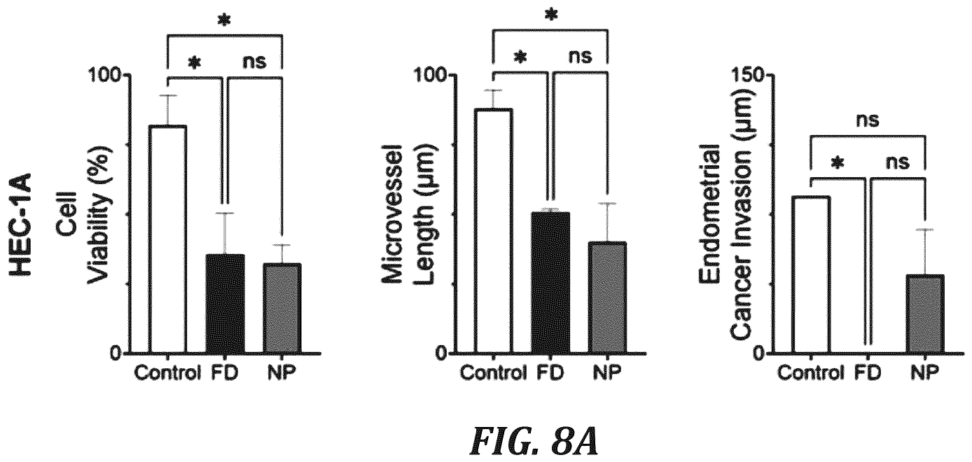

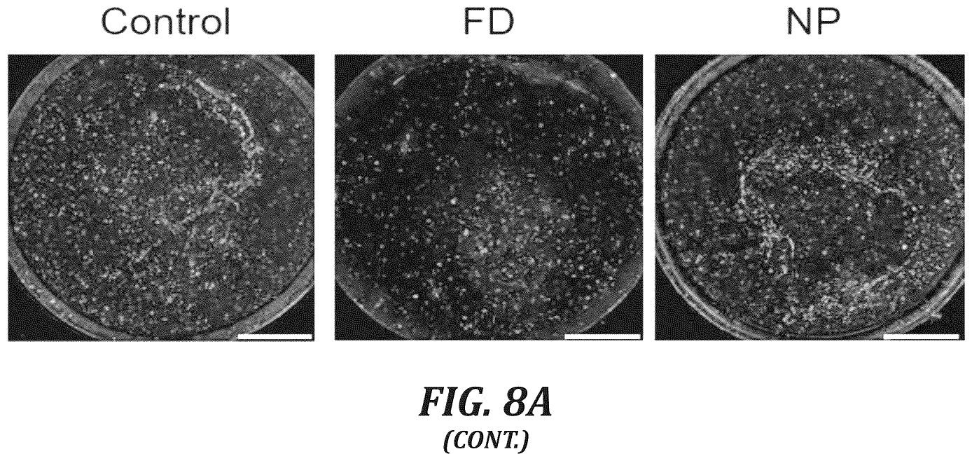

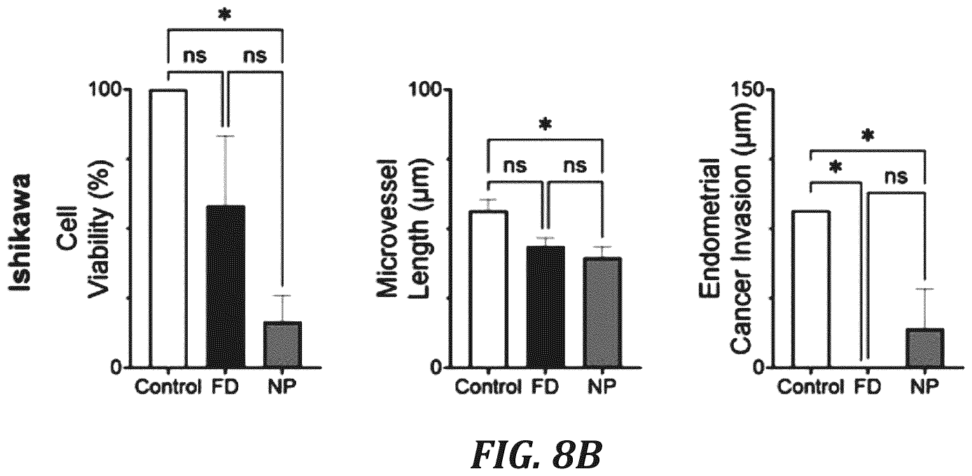

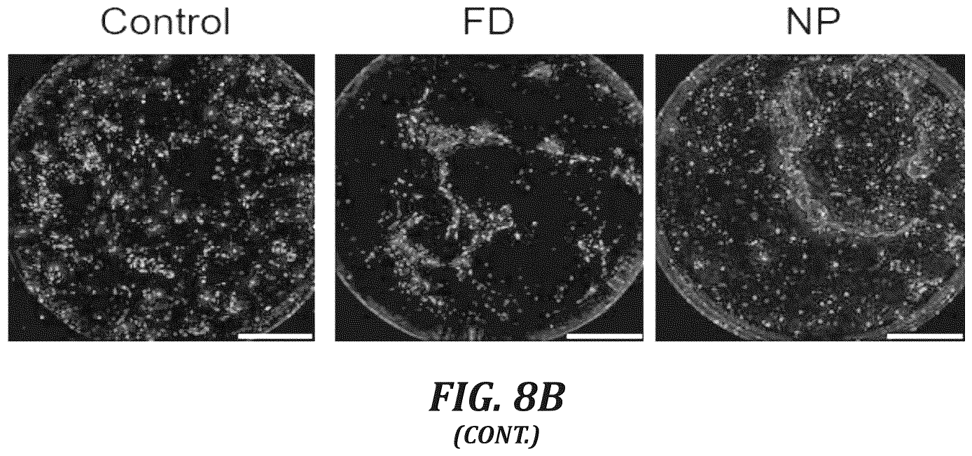

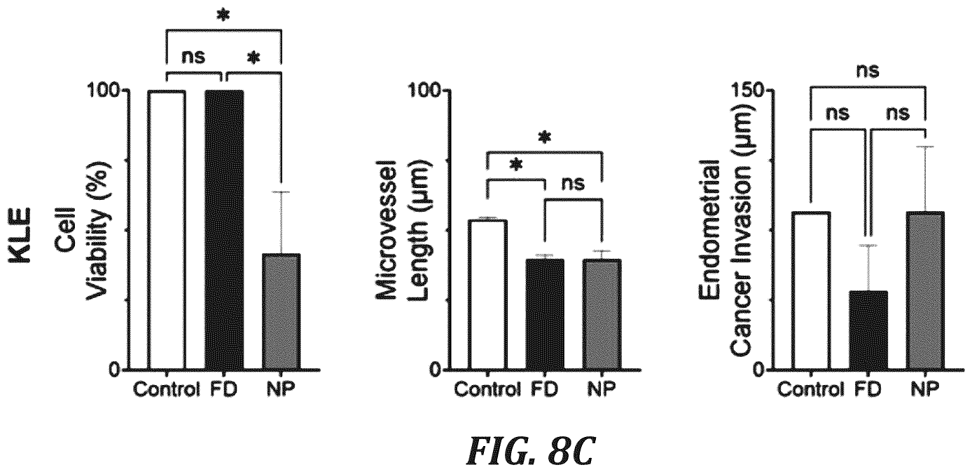

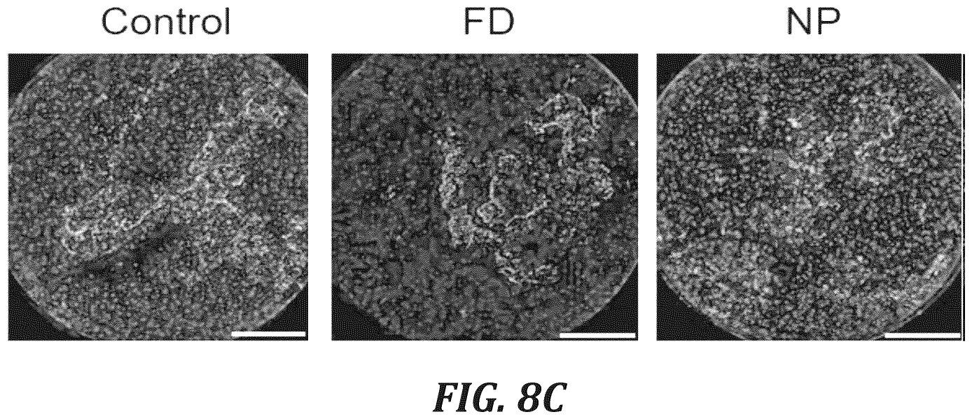

Constructs with HEC-1A cells were treated with 4.02 μM of paclitaxel, either as a free drug or loaded in PCL nanoparticles. Both delivery methods resulted in decreased viability and phenotypic responses. Paclitaxel, dispensed as a free drug, was more effective in reducing endometrial cancer invasion. There were no significant differences between the dosing methods; however, both methods showed reduced cell viability and phenotypic cell responses compared to the control group (FIG. 8A). 3D cultures with Ishiwaka cells were dosed with paclitaxel at a concentration of 4.34 μM for both free drug and PCL nanoparticles. Similar to constructs with HEC-1A cells, there was a reduction in cell viability with the PCL nanoparticles, and invasion was decreased with the free drug. Again, there were no significant differences between free drug and PCL nanoparticles. However, only the PCL nanoparticles showed significant differences in the reduction of cell viability compared to the control group (FIG. 8B).

The KLE cell models were treated with paclitaxel at 8.78 μM, revealing the highest resistance among all the evaluated cancer cell lines in both cell viability and endometrial cancer invasion (FIG. 8C). Notably, significant differences emerged between the two dosing methods in terms of cell viability. Specifically, only the PCL nanoparticles effectively reduced cell viability, exhibiting significant differences compared to the control group. In contrast, both treatments resulted in a similar decrease in microvessel length, with significant differences only observed compared to the control group. Moreover, the impact on endometrial cancer invasion was solely observed in response to the free drug.

### SUMMARY

Using Design of Experiments (DOE), a multilayer multicellular model was developed that replicates the complex cell responses of endometrial cancer within a 96-well format, making it suitable for high-throughput screening. The model is a hydrogel system that consists of two specialized hydrogel layers designed to support both endothelial (hMVEC) and endometrial cancer (HEC-1A) cells, enabling crosstalk that mimics interactions within the tumor microenvironment. Additionally, this system was tested with two other endometrial cancer cell lines (Ishikawa and KLE), allowing investigation of distinct responses of these lines to paclitaxel, both in its free form and encapsulated in PCL nanoparticles. This model revealed nuanced cell behaviors that are not captured by conventional 2D cell culture or endpoint assays.

The DOE approach was used to develop hydrogel formulations aimed at maximizing endometrial cancer cell invasion and endothelial cell microvessel formation simultaneously. To achieve this, a multilinear regression model was used with D-optimal design, which generated linear and quadratic effects for input variables, including Col IV, FN, LMN, FG, and Col I, on four key responses: microvessel length, endometrial cancer invasion, endothelial cell coverage, and cancer cell coverage. D-optimal design is well-suited for studies that include both categorical and continuous variables, as it allowed us to vary ECM component concentrations and select either PEGDA or GelMA as the synthetic polymer in the hydrogel formulations. The observed effects of the input variables across the top and bottom layers served as a guide to identify optimal conditions where microvessel formation and cancer invasion co-exist at maximal levels—critical targets for drug screening. Using a prediction tool, three design points were evaluated that maximized microvessel length, cancer invasion, and cell coverage, while minimizing failure probability. These experiments lead to representative hydrogel systems that maximized microvessel formation, endometrial cancer invasion, and cell coverage.

The two distinct formulations for the top (cancer epithelial) and bottom (endothelial) layers incorporated a blend of ECM components and synthetic polymers, resulting in enhanced metastatic behaviors compared to the traditional model of Matrigel. Specifically, the bottom hydrogel layer was developed using a blend of Col I, fibrin, and synthetic polymer GelMA. Varying Col I and fibrin concentrations between 0.5 and 2.5 mg/mL significantly impacted microvessel formation and cancer invasion, with 1.91 mg/mL Col I and 0.6 mg/mL fibrin maximizing these responses, demonstrating significant interactions. For the top layer, a combination of Col IV, fibronectin, laminin, and PEGDA was used, addressing the need for 3D in vitro models that better replicate the ECM in the endometrial cancer microenvironment. The importance of co-culture was confirmed by comparing systems with both cell types to monocultures. Certain hydrogel formulations (Max All) outperformed Matrigel, showing increased microvessel length and cancer invasion in coculture models. These outcomes highlight the critical role of cancer-endothelial cell communication, which, alongside physical cues from the microenvironment, plays a role in influencing both of these phenotypes within the tumor microenvironment.

Recent studies underscore the need for 3D in vitro models that better simulate the tumor microenvironment in endometrial cancer, surpassing the limitations of traditional 2D cultures and 3D models built on Matrigel. The hydrogel system described herein is a platform to address this gap by incorporating key ECM components alongside synthetic polymers, optimizing the hydrogel's ability to support both endothelial and cancer cell behaviors. Col IV, a predominant structural component of cellular basement membranes, plays a complex, context-dependent role in endometrial cancer progression. As described herein, a concentration of 0.13 mg/mL Col IV maximized cancer invasion, which is an order of magnitude lower than the 2.8 mg/mL found in Matrigel. Similarly, laminin, known for its role in cell adhesion and migration, maximized cancer invasion at 0.5 mg/mL laminin, also significantly lower than Matrigel's 5.5 mg/mL. These lower optimal concentrations may explain why Matrigel induced limited endometrial cancer invasion. Fibronectin, another critical ECM component of the endometrial tumor microenvironment that promotes cancer growth and invasion, further enhanced metastatic behaviors at 0.17 mg/mL fibronectin, interacting significantly with both Col IV and laminin. Together, these ECM components form a more accurate representation of the tumor microenvironment, crucial for studies on cancer progression and drug screening. Additionally, the incorporation of PEGDA in the top layer and GelMA in the bottom provided mechanical flexibility needed to support both cell types while maintaining the structural integrity of the coculture system.

Matrigel is commonly used as a control in 3D culture systems due to its ability to promote epithelial attachment and differentiation and its historic role in cancer biology. However, it is significantly softer than most tumor microenvironments, including those of the endometrium, which limits its ability to replicate the mechanical conditions critical for tumor progression. The viscoelastic properties of the tumor microenvironment are known to influence cell migration, invasion, and vasculogenesis, underscoring the need for models that more closely mimic these mechanical characteristics. As described herein, the viscoelastic properties of representative hydrogels and Matrigel were measured to assess how well they replicate the native endometrium. The results demonstrated that the top hydrogel formulation had a storage modulus (G′) of 916 Pa and a loss modulus (G″) of 115 Pa. While this stiffness exceeds reported values for nonpregnant endometrial tissue (elastic modulus 250 Pa) and uterine tissue (complex modulus 100 Pa), it nonetheless represents a substantial improvement over commonly used matrices such as Matrigel, which exhibited a much lower stiffness (66 Pa G′, 9 Pa G″), The increased stiffness of the hydrogels, attributed to the incorporation of Collagen I alongside PEGDA or GelMA, supports better structural integrity and mechanical resistance for in vitro tumor modeling. Although the hydrogel is stiffer than native tissue, its properties may be better suited for modeling the altered mechanical microenvironment observed in solid tumors, as clinical studies have demonstrated that increased stiffness in the tumor microenvironment correlates with enhanced cancer invasion and progression. By incorporating materials that better represent the mechanical environment of the endometrium, the hydrogel system described herein addresses the limitations of Matrigel and provides a more realistic platform for studying endometrial cancer invasion and drug responses.

To validate the representative hydrogel systems across different stages of endometrial cancer, three endometrial cancer cell lines were evaluated: HEC-1A (stage 1 endometrial carcinoma), Ishikawa (stage 2, endometrial adenocarcinoma), and KLE (metastatic adenocarcinoma). Given the stage and grade of these cell lines at the time of isolation, distinct phenotypic behaviors for each cell line were anticipated. Over a 48-hour period, cancer cell invasion increased for all three cell lines, indicating that the hydrogels successfully supported the in vivo behavior of cancer cells in a 3D environment, which is often lost in 2D cultures. Interestingly, despite being associated with early-stage disease, HEC-1A cells exhibited the highest invasiveness, providing new insights into how these cell lines interact with endothelial cells in a 3D co-culture. Moreover, across all cell lines, an inverse relationship between cancer cell coverage and endothelial cell coverage was observed: as cancer cell coverage increased, endothelial cell coverage decreased. Given that these cells reside in different layers of the 3D representative hydrogel system, this interaction was particularly notable. Previous studies using spheroid models have demonstrated that retaining in vivo behavior is crucial for driving accurate phenotypic responses, supporting our observations in this 3D system.

When treated with paclitaxel, the representative hydrogel system described herein revealed significant differences in drug sensitivities between the three cell lines, which contrasted sharply with results observed in 2D culture. As expected, HEC-1A cells, known for their paclitaxel resistance, exhibited the highest resistance, while KLE cells, known to be paclitaxel-sensitive, showed the greatest sensitivity. Previous reports have also shown that Ishikawa cells are even less sensitive to paclitaxel than HEC-1A cells. the representative hydrogel system described herein supported these findings, with HEC-1A demonstrating the most resistance and KLE the most sensitivity. Interestingly, the IC₅₀ curves had a better fit for endometrial cancer invasion and endothelial microvessel formation compared to cell viability. Microvessel formation followed the expected IC₅₀ trends for paclitaxel-sensitive versus paclitaxel-insensitive cancer cells in co-culture, while cancer invasion exhibited an opposite trend, potentially due to the influence of microvessel dynamics. These observations underscore the importance of capturing both ECM behavior and cell crosstalk in assessing drug responses. Furthermore, the IC₅₀ values derived from 2D cultures did not align with the known paclitaxel sensitivities of these cell lines while the 3D models more accurately predicted in vivo behavior, consistent with previous findings. Finally, the IC₅₀ curves for cell coverage showed a poor fit, suggesting that this parameter is not a reliable predictor of paclitaxel sensitivity in vivo. Overall, these findings emphasize the need to dissect specific phenotypic responses, as different endometrial cancer cell lines exhibit varied susceptibilities to paclitaxel within the 3D microenvironment.

Paclitaxel is a widely used chemotherapeutic agent for the treatment of endometrial cancer, but its intravenous administration is often limited by poor solubility and systemic toxicity, reducing its overall therapeutic efficacy. To address these challenges, a poly(caprolactone) (PCL) nanoparticle delivery system was used for paclitaxel delivery that was designed to improve solubility and provide a sustained-release mechanism that reduces systemic toxicity while enhancing drug bioavailability at the tumor site (S. O. Alhaj-Suliman, E. I. Wafa, A. K. Salem, Adv Drug Deliv Rev 2022, 189, 114482). Nanoparticle-based delivery systems, such as nanoparticle albumin-bound paclitaxel (nab-paclitaxel), have already shown clinical promise in the treatment of breast and pancreatic cancers. However, PCL nanoparticles offer additional benefits. The sustained-release properties of the PCL nanoparticles allow paclitaxel to diffuse gradually into the tumor microenvironment, improving local drug concentrations while minimizing toxicity. Furthermore, the PCL nanoparticles enhance cancer cell sensitivity to paclitaxel by facilitating endocytosis, thereby increasing intracellular drug levels and enabling lower doses to achieve therapeutic effects.

To evaluate the efficacy of PCL-encapsulated paclitaxel, the IC₅₀ values of three endometrial cancer cell lines (HEC-1A, KLE, and Ishikawa) cultured in 2D on tissue culture plastic over 24 or 48 hours was measured. The results demonstrated that PCL-encapsulated paclitaxel significantly lowered IC₅₀ values in HEC-1A and KLE cells compared to the free drug, indicating enhanced drug efficacy in these lines. In contrast, the IC₅₀ values for Ishikawa cells when treated with PCL-encapsulated paclitaxel compared to the free drug, which may be attributed to the narrow therapeutic window for paclitaxel in this cell line. A plateau and subsequent increase in cell viability at higher doses was observed, suggesting that the optimal dosing range for paclitaxel delivery may vary across different endometrial cancer subtypes. To further assess the effects of PCL-encapsulated paclitaxel in the representative hydrogel system (3D co-culture model), the relative viability of untreated controls, free paclitaxel, and PCL-encapsulated paclitaxel was compared. While encapsulation did not significantly decrease cell viability compared to free paclitaxel in HEC-1A and Ishikawa cells, a marked decrease in viability was observed in KLE cells, which are known to be more sensitive to paclitaxel. This suggests that the sensitivity of KLE cells likely enhances their response to the nanoparticle-form of paclitaxel.

For paclitaxel-resistant cell lines, no significant differences in cell viability were observed between free and PCL-encapsulated paclitaxel. However, both treatments showed significant differences compared to untreated controls, consistent with previous studies demonstrating the efficacy of polymeric nanoparticles in drug delivery. This trend was also observed in microvessel formation across all three cell lines. Paclitaxel had limited effects on cancer cell invasion, with the exception of the Ishikawa cell line, where free paclitaxel abrogated all cancer invasion. These results highlight the importance of using a co-culture model to capture multiple facets of endometrial cancer metastasis beyond cell viability. Furthermore, this approach aligns with the FDA's recent recognition of the value of 3D models in preclinical data for drug development. The 3D model represents a significant advancement in identifying new therapeutic targets, evaluating drug efficacy, and supporting personalized medicine for endometrial cancer patients.

### Materials and Methods

### Cell Lines and Reagents

Unless otherwise stated, all reagents were purchased from ThermoFisher (Waltham, MA). Human microvascular endothelial cells (hMVEC) were purchased from Lonza (hMVEC 33226, Walkersville, MD) and used without additional characterization. Cells were expanded in EGM-2 MV media (EBM-2 supplemented with Lonza's SingleQuot supplements: hydrocortisone, human basic fibroblast growth factor (FGF2), human vascular endothelial growth factor (VEGF), human insulin-like growth factor (IGF), human epidermal growth factor (EGF), ascorbic acid, and gentamycin) and further supplemented with 5% fetal bovine serum (FBS) until used at passage 5. Human endometrial cancer cell lines HEC-1A (ATCC HTB-112™) and KLE (ATCC CRL-1622) and the human cervical cancer cell line SiHa (ATCC® HTB-35™) were purchased from ATCC (Manassas, VA) and used without additional characterization. The cells were cultured in McCoy's 5A medium (HEC-1A, ATCC), DMEM: F12 medium (KLE, ATCC), and Eagle's Minimum Essential Medium (SiHa, EMEM, ATCC), supplemented with 1% penicillin-streptomycin (Sigma-Aldrich, St. Louis, MO, USA) and 10% FBS. Ishikawa H cells were obtained from Dr. Aliasger Salem (University of Iowa, Iowa City, IA). Ishikawa cells were cultured in DMEM medium (Quality Biological, Gaithersburg, MD) supplemented with 10% FBS and 1% penicillin-streptomycin. HEC-1A and KLE cells are categorized as Type II endometrial cancer cell lines, known for their highly aggressive and invasive cancer characteristics. In contrast, Ishikawa cells are identified as a Type I endometrial cancer cell line, characterized by low histological differentiation, representing an early-stage disease. Furthermore, the HEC-1A and the Ishikawa cell lines are considered paclitaxel-insensitive, whereas KLE cells are considered paclitaxel-sensitive. All cell types were expanded in standard cell culture conditions (37° C., 21% 02, 5% CO₂) and subcultured before they reached 80% confluency.

### Multilayer Hydrogel Fabrication

The hydrogel system described herein, a multilayer multicellular 3D model of endometrial cancer is illustrated schematically in FIG. 1A. To prepare the bottom hydrogel supporting endothelial cells (hMVEC), stock solutions of bovine collagen type I (10 mg/ml, FibriCol, Advanced BioMatrix), 1 N NaOH, fibrinogen from human plasma (30 mg/mL Sigma-Aldrich), 50 U/mL thrombin (20 U, Sigma-Aldrich) and either polyethylene glycol diacrylate (PEGDA) (11.7% w/v, Advanced BioMatrix, Carlsbad, CA, USA) or gelatin methacryloyl (GelMA) (8.7% w/v, Advanced BioMatrix) were mixed in PBS and photo-crosslinked using a CL-1000 UVP crosslinker with irgacure 2959 (365 nm) as specified by the manufacturer (10% w/v, Advanced BioMatrix). To formulate the top hydrogel, stock concentrations of laminin (1 mg/mL, Sigma-Aldrich), human fibronectin (1 mg/mL, Advanced Biomatrix), collagen IV (1 mg/mL, Advanced Biomatrix), 1 N NaOH, and either GelMA (8.7% v/w) or PEGDA (11.7% w/v) were mixed in PBS. The construct was photo-crosslinked for 30 seconds in a CL-1000 UVP crosslinker using the same conditions as above.

Photocrosslinking to form the hydrogel is carried out after the addition of each hydrogel formulation. For the bottom hydrogel, the photocrosslinking was for 1 min. After the addition of the top hydrogel formulation, the photocrosslinking was for 30 seconds, as endothelial cells are between the top and bottom hydrogels, and a long exposure to UV light can damage them. No effects in cell response or viability was observed using a UV exposure of 30 seconds.

Constructs were fabricated in specialized p-Plate Angiogenesis 96-wells (ibidi, Munich, Germany). First, 10 μL of the bottom hydrogel formulation was loaded into each well and the plate was incubated for one hour at 37° C. After one hour, 9,400 CellTracker Green labeled hMVEC cells in 40 μL of EGM-2 MV were pipetted on top of each hydrogel. The hMVEC endothelial cells were allowed to attach for four hours at 37° C. and 5% CO₂. The media was removed, and 25 μL of the top hydrogel formulation was pipetted on top of the endothelial cells and the plate was incubated for one hour at 37° C. Finally, CellTracker Red labeled endometrial cancer cells were seeded on top of each gel at 12,500 cells in 25 μL of media. Cells were then maintained in a 1:1 ratio of EGM-2 MV and cancer cell medium, dependent upon cell type. As a control, multilayer Matrigel constructs were prepared using the same methods described above except replacing the custom hydrogel formulations with Growth Factor Reduced Basement Membrane Matrigel in both layers (9.2 mg/mL protein concentration, Corning, MA, USA).

### Evaluation of Phenotypic Cell Response

All reagents were purchased from ThermoFisher (Waltham, MA) unless stated otherwise. Phenotypic cell responses were monitored by staining endothelial cells, endometrial cancer cells, and cervical cancer cells with fluorescent dyes, CellTracker Green CMFDA (C7025), CellTracker Blue CMF2HC (C12881), CellTracker Red CMTPX (C34552) or CellTracker Deep Red (C34565), before the experiment. For all experiments, two-channel z-stack images with 85 μm or 95 μm z-slice spacing (specific spacing is described on each figure capture). A 4× objective with a depth of field at 61 μm was used to image the 3D models. Images were taken every 3 or 6 hours for 48 hours using a Cytation 5-cell imaging multimode reader (Agilent Technologies). From these images, Z-projected images were created using the integrated Gen 5 software (Agilent Technologies). Using a focus stacking with a maximum filter size of 11 px, we captured the cell response in one 2D image. Images were processed with Fiji (NIH, Bethesda, MD), and cell coverage was measured for each cell type at every time point by calculating the area within the well covered by cells based on fluorescent labeling and dividing it by the total well area. A macro recorded code was generated to process a batch of images over time. Cell coverage was reported as a percentage of the endothelial cells or the percentage of cancer cells in the well as appropriate. Cell invasion depth was measured from the z-stack images using the integrated Gen 5 software (Agilent Technologies), and invasion depth was defined as the image where at least 80% of the cells remained in focus. Endothelial microvessel formation was manually quantified by measuring the average microvessel length at each time point with Fiji. See FIGS. 1A-1C.

To evaluate cell morphology in 2D culture, confocal microscopy images were taken for endometrial cancer cell lines seeded on 0.01% poly(L-lysine) (PLL)-coated coverslips (Sigma Aldrich, St. Louis, MO). Cells were incubated for 24 hours prior to fixation with 4% paraformaldehyde for 10 minutes, followed by 0.1% Triton-X (Research Products International, Corp) in 1×DPBS for five minutes. Non-specific binding was blocked with 10 mg/mL bovine serum albumin (BSA, Sigma Aldrich) in 1×DPBS for 20 minutes. Cells were stained with 0.165 μM AlexaFluor488-phalloidin (ThermoFisher) for 20 minutes and then mounted onto coverslips using ProLong Diamon Anitfade Mountant with DAPI (ThermoFisher) sealed with clear nail polish. Cells were imaged on an Upright Zeiss LSM 880 multi-photon microscope.

Cell viability was quantified using cell titer-glo 3D cell viability assay (Promega, Madison, WI). Following the manufacturer's instructions, the reagent was added on top of the wells at either 24 or 48 hours post dosage of the chemotherapy agent, paclitaxel. Cell metabolic activity was quantified in 2D models using an MTT assay (Thermo Fisher, Waltham, MA). For the MTT assays, endometrial cancer cell lines were seeded at 1.5×10⁴ cells/well in a 96-well plate. The cells were incubated overnight before adding the chemotherapy agent as free paclitaxel or PCL particles loaded with paclitaxel. After 24 or 48 hours, the media was removed and replaced with 100 μL of media and 10 μL of 5 mg/mL MTT in 1× Dulbecco's Phosphate Buffered Saline (DPBS). The cells were incubated for four more hours. Finally, the media was removed and 100 μL of an isopropanol:dimethyl sulfoxide (IPA:DMSO, 9:1) solution was added to stop the reaction. Absorbance was measured at 570 nm on a BioTek Synergy H1 plate reader, and cell viability was calculated as a percentage of the untreated control.

### Design of Experiments

The significance and interactions of the concentrations of natural (collagen type I and IV, fibrin, fibronectin, laminin) and synthetic (GelMA, PEGDA) polymers present in each hydrogel layer on the function of each cell type was determined. A D-Optimal design with six quantitative factors and two qualitative factors was created with MODDE Pro software version 12.1 (Sartorius AG, Göttingen, Germany). The five quantitative factors were (1) 0.5 to 2.5 mg/mL collagen type I in the bottom hydrogel, (2) 0.5 to 2.5 mg/mL fibrin in the bottom hydrogel (3) 0.01 to 0.2 mg/mL collagen type IV in the top hydrogel, (4) 0.125 to 0.175 mg/mL fibronectin in the top hydrogel, and (5) 0.5 to 2 μg/mL laminin in the top hydrogel. All polymers were diluted to the desired concentration with 1×PBS. The two qualitative factors were PEGDA (10% w/v) or GelMA (7% w/v) in both top and bottom hydrogel layers. The four output variables were (1) endothelial cell microvessel length, (2) endothelial cell coverage, (3) endometrial cancer invasion depth, and (4) endometrial cancer cell coverage. Models were developed and validated using only the HEC-1A cell line, as they could be used for further drug sensitivity studies. The D-Optimal design generated 45 unique hydrogel combinations with a centrally repeated condition to optimize the Design's G-efficiency, which assessed how well the D-Optimal design compared to a fractional factorial design. The significance and interaction of all the input variables on the measured output variables were evaluated based on the R² and Q² diagnostics of the fit. The model was validated by evaluating the hydrogel formulations that were predicted to either maximize all or minimize all four of the phenotypic cell responses simultaneously. The multilayer multicellular platform that achieved the engineering objective of maximizing all of the output variables simultaneously is referred to as the representative hydrogel system described herein (Table 3).

TABLE 3
Hydrogel formulations predicted to maximize or minimize cell phenotypes
of interest in the bottom hydrogel (B) or top hydrogel (T). FG =
fibrin, Syn = synthetic polymer, FN = fibronectin, LMN = laminin.

| | [Col I]_{B} | [FG]_{B} | | [Col IV]_{T} | [FN]_{T} | [LMN]_{T} | |
|---|---|---|---|---|---|---|---|
| Response | (mg/mL) | (mg/mL) | [Syn]_{B} | (mg/mL) | (mg/mL) | (μg/mL) | [Syn]_{T} |
| Maximize | 1.91 | 0.60 | GelMA | 0.13 | 0.17 | 0.50 | PEGDA |
| all cell | | | | | | | |
| phenotypes | | | | | | | |
| of interest | | | | | | | |
| (Optimized | | | | | | | |
| endometrial | | | | | | | |
| cancer model) | | | | | | | |
| Minimize | 0.70 | 0.70 | PEGDA | 0.03 | 0.16 | 0.65 | PEGDA |
| all cell | | | | | | | |
| phenotypes | | | | | | | |
| of interest | | | | | | | |

### Co-Culture and Monoculture Experiments

To evaluate the influence of crosstalk between endothelial cells and endometrial cancer cells in coculture, the representative hydrogel (endometrial cancer model) was fabricated with endometrial cancer cells alone, endothelial cells alone, or a co-culture of both endometrial cancer and endothelial cells. A multilayer Matrigel platform was used as a control, replacing both of the optimized hydrogel formulations. All monoculture and co-culture experiments included both the top and the bottom hydrogel layers. Cell phenotypic responses were measured over 48 hours after seeding cells according as described above. Differences between the monoculture and co-culture models were assessed from differences in microvessel formation, invasion depth, and cell area.

### Assessment of Material Properties

Dynamic oscillatory shear measurements were used to evaluate the rheological properties of the bottom and top hydrogels of the optimized hydrogel formulation, as well as GFD Matrigel. The bottom and top hydrogels with GelMA or PEGDA were photocrosslinked with 365 nm UV light and incubated at 37° C. before rheological testing. An AR-G2 rheometer (TA Instruments, New Castle, DE) equipped with a 20 mm standard steel parallel top plate and a bottom standard Peltier plate was used to conduct rheological characterization of the hydrogels. Due to the tendency for the gels to slip, 150 grit (120 μm particle size) sandpaper adhered to both the top and bottom fixtures. The gap was adjusted between measurements to ensure contact between the top plate and the gel such that the normal force was 0.1 N. All measurements were performed at a constant temperature of 37° C. using a raised Peltier plate control system. Frequency sweeps were conducted from 0.1 to 10 rad/sec within the linear viscoelastic (LVE) region at a constant strain amplitude of 5%. The storage moduli (G′) and loss moduli (G″) were measured across the frequency range, the average of the linear region was calculated for each hydrogel, and three consecutive technical replicates were conducted for each hydrogel formulation. The gels were allowed to equilibrate to the plate temperature of 37° C. for 15 minutes, at which point frequency sweeps were conducted.

### Paclitaxel-Loaded Nanoparticles Preparation

Paclitaxel-loaded poly(caprolactone) (PCL) nanoparticles were prepared using a previously reported method based on an oil-in-water emulsion solvent evaporation (A. N. Manning, C. E. Rowlands, H. Saindon, B. E. Givens, AAPS J 2023, 25, 100). The oil phase consisted of 60 mg of PCL (Mn 80,000, Sigma Aldrich, Milwaukee, WI), 3 mg of paclitaxel (Selleck Chemicals LLC, Houston, TX), and 3 mL of dichloromethane (DCM) (VWR, Radnor, PA). The water phase consisted of a 2.5% (w/v) of poly (vinyl alcohol) (PVA) (87-90% hydrolyzed, average molecular weight 30,000-70,000, Sigma-Aldrich, Milwaukee, WI) in deionized water, which was stirred for 20 minutes to allow the PVA to dissolve. The oil phase was prepared by mixing PCL, paclitaxel, and DCM for 15 minutes until PCL and paclitaxel were dissolved and vortexed prior to sonication. Under probe sonication at 50% power for 75s (Q500 Sonicator, QSonica, Newton, CT), the oil phase was added slowly dropwise directly onto the probe into 50 mL of the water phase. After sonication, the emulsion was added to 30 mL of the water phase (total volume=83 mL) and stirred at 350 rpm for 30 minutes to evaporate the DCM. The solution was then centrifuged at 200 rcf to remove unreacted PCL, and the supernatant was then centrifuged at 1000 rcf at 20° C. for 10 minutes. The pellet formed at 1000 rcf was discarded; the supernatant was collected and spun at 3000 rcf for 10 minutes. The pellet formed at 3000 rcf was washed three times with DI water at 3000 rcf to remove the PVA. After washing, the particles were suspended in DI water and frozen at −20° C. then lyophilized (FreezeZone Bench Top freeze dryer, Labconco, Kansas City, MO) for at least 24 hours or until only a fine powder remained. The particles were stored at −20° C. until use.

### Dose-Response Analysis of Paclitaxel

To compare free-drug, obtained from the Oregon State University College of Pharmacy High-Throughput Screening Services Laboratory (HTSSL), and PCL-loaded paclitaxel, a dose-response analysis was conducted of paclitaxel in 2D cultures with endometrial cancer cells seeded onto tissue culture plastic and 3D culture models with endometrial cancer cells seeded onto the optimized hydrogel model.

For free drug analysis in 2D, endometrial cancer cells (1.5×10⁴ cells/well) were seeded in 96-well plates with 35 μL of endometrial cancer culture media. After 24 hours of culturing, we performed an eight-point half-log screen ranging from 0.008 to 25 PM. Paclitaxel was diluted with dimethyl sulfoxide (DMSO) and dispensed onto the wells using an automatic liquid handler (D300e Digital Dispenser, Hewlett Packard [HP], Corvallis, OR). Media alone and DMSO controls were also evaluated. The IC₅₀ values were evaluated for each cell line using the conventional metric of concentration that reduces the cell viability by 50%. Specifically, the luminescence of cell titer glo or absorbance of MTT values were normalized to the average of the values observed in the untreated control, and each replicate was considered an individual point. The IC₅₀ and Hill slope were determined from these results (Equation 1). The dose response-inhibition curves were calculated using Prism 8.2.1 software (GraphPad, San Diego, CA).

$$Y = \frac{100}{1 + {10}^{( ( {LogIC}_{50} - X ) \times H illSlope )}} \qquad Equation 1$$

where Y is the normalized cell response, and X is the log of the drug concentration.

The IC₅₀ values were then evaluated in terms of phenotypic responses. Using the representative hydrogel systems, CellTracker blue-labeled endothelial cells and CellTracker green-labeled endometrial cancer cells were seeded in the optimized construct described above and cultured for 24 hours. After 24 hours of culturing, an eight-point half-log screen was performed by dispensing paclitaxel on top of the 3D in vitro models with the automatic liquid handler (D300e, HP) and using DMSO as a vehicle. Paclitaxel loaded in PCL nanoparticles was pipetted by hand into the wells at the same concentration of the free drug. Phenotypic cell responses were measured after 24 and 48 hours of treatment. The IC₅₀ response curves were then calculated using Equation 1.

### Statistical Analysis

Statistical analyses were performed using a two-way analysis of variance (ANOVA) with Tukey correction for multiple comparisons or one-way ANOVA when appropriate. Dunnet correction was used in the DOE comparison to account for the significant difference between the predicted hydrogels and Matrigel. All statistical analyses were performed using Prism 8.2.1 software (GraphPad, San Diego, CA); p-values less than 0.05 were considered statistically significant. Replicates are specified in each experiment, and asterisks denote statistical significance.

As used herein, the term “about” refers to ±5% of the specified value.

While illustrative embodiments have been illustrated and described, it will be appreciated that various changes can be made therein without departing from the spirit and scope of the invention.

The embodiments of the invention in which an exclusive property or privilege is claimed are defined as follows:

1. A hydrogel system, comprising:
(a) a first hydrogel having a first surface and an opposing second surface, wherein the first hydrogel is a crosslinked gelatin hydrogel comprising collagen I and fibrin;
(b) a second hydrogel having a first surface and an opposing second surface, wherein the second hydrogel is a crosslinked poly(ethylene glycol) hydrogel comprising collagen IV, fibronectin, and laminin, wherein the second surface (bottom) of the second hydrogel (top) is adjacent the first surface of the first hydrogel;
(c) endothelial cells intermediate and substantially coextensive with the first surface of the first hydrogel and the second surface of the second hydrogel; and
(d) endometrial cancer cells disposed on the first surface of the second hydrogel.

2. The hydrogel system of claim 1, wherein the endothelial cells and cancer cells are in biological communication (cell crosstalk) through the second hydrogel.

3. The hydrogel system of claim 1, wherein collagen I is present in the first hydrogel in an amount from about 10 to about 15 percent of the total volume of the first hydrogel.

4. The hydrogel system of claim 1, wherein fibrin is present in the first hydrogel in an amount from about 10 to about 15 percent of the total volume of the hydrogel.

5. The hydrogel system of claim 1, wherein collagen IV is present in the second hydrogel in a concentration from about 0.10 to about 0.20 mg/mL.

6. The hydrogel system of claim 1, wherein fibronectin is present in the second hydrogel in a concentration from about 0.12 to about 0.18 mg/mL.

7. The hydrogel system of claim 1, wherein laminin is present in the second hydrogel in a concentration from about 0.5 to about 2 μg/mL.

8. The hydrogel system of claim 1, wherein the second hydrogel has storage modulus (G′) from about 900 to about 1000 Pa.

9. The hydrogel system of claim 1, wherein the second hydrogel has storage loss modulus (G″) from about 100 to about 150 Pa.

10. The hydrogel system of claim 1, wherein the first hydrogel has storage modulus (G′) from about 100 to about 200 Pa.

11. The hydrogel system of claim 1, wherein the first hydrogel has storage loss modulus (G″) from about 20 to about 100 Pa.

12. A method for assessing the effectiveness of a therapeutic agent for the treatment of endometrial cancer, comprising:
(a) contacting a hydrogel system of claim 1 having endometrial cancer cells in the second hydrogel; and
(b) determining the viability of the cancer cells after a predetermined period of time to assess the effectiveness of the therapeutic agent for the treatment of endometrial cancer.

13. The method of claim 12, further comprising determining the cancer cell invasion depth after the predetermined period of time.

14. The method of claim 12, further comprising determining the reduction of endothelial microvessel length after the predetermined period of time.
